(19) United States
(12) Patent Application Publication
(10) Pub. No.: US 2018/0078532 A1
(43) Pub. Date: Mar. 22, 2018

(54) IMMEDIATE RELEASE FORMULATIONS FOR OPROZOMIB

(71) Applicant: AMGEN INC., THOUSAND OAKS, CA (US)

(72) Inventors: John Inn CHUNG, Porter Ranch, CA (US); Armen PIRJANIAN, Sunland, CA (US); Fernando ALVAREZ-NUNEZ, Newbury Park, CA (US); Jeffrey Michael KATZ, Oak Park, CA (US); Dominick DAURIO, Camarillo, CA (US)

(73) Assignee: AMGEN INC., Thousand Oaks, CA (US)

(21) Appl. No.: 15/707,005

(22) Filed: Sep. 18, 2017

Related U.S. Application Data
(60) Provisional application No. 62/397,830, filed on Sep. 21, 2016.

(51) Int. Cl. A61K 31/426; A61K 47/02; A61K 47/38; A61K 47/12; A61K 9/20; A61K 9/28

(52) U.S. Cl. CPC A61K 31/426; A61K 47/02; A61K 47/38; A61K 9/28; A61K 9/2018; A61K 9/2054; A61K 9/2009; A61K 47/12

(57) ABSTRACT

This disclosure features immediate release pharmaceutical formulations (e.g., solid dosage forms, e.g., tablets) that are useful for the oral administration of oprozomib, or a pharmaceutically acceptable salt thereof, to a human or animal subject as well as methods of making and using the formulations.

This application claims the benefit of U.S. Provisional Application No. 62/397,830, filed Sep. 21, 2016, which is hereby incorporated by reference in its entirety for all purposes.

### TECHNICAL FIELD

This disclosure features pharmaceutical formulations (e.g., immediate release (IR) pharmaceutical formulations; e.g., solid dosage forms, e.g., tablets) that are useful for the oral administration of oprozomib, or a pharmaceutically acceptable salt thereof, to a human or animal subject as well as methods of making and using the formulations. A tablet composition and process was developed that produces an immediate release tablet that disintegrates quickly, thus allowing for absorption in the duodenum and jejunum regions of the gastrointestinal tract, thereby decreasing the adverse effects of nausea, vomiting and/or diarrhea.

### BACKGROUND

The proteasome has been validated as a therapeutic target, as demonstrated by the FDA approval of bortezomib, a boronic acid proteasome inhibitor, for the treatment of various cancer indications, including multiple myeloma; and more recently, carfilzomib, a tetra-peptide epoxy ketone-containing proteasome inhibitor, for the treatment of refractory multiple myeloma.

Oprozomib (chemical structure shown below) is an orally bioavailable (epoxy ketone-containing) tri-peptide irreversible proteasome inhibitor, which has demonstrated preclinical anti-tumor activity and a broad therapeutic window in preclinical models and is currently being studied in Phase I clinical trials.

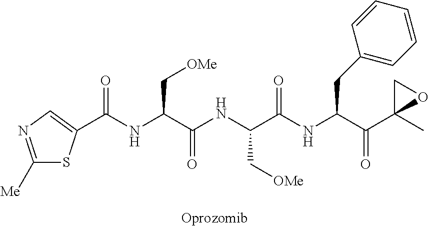 [SMILES: COC[C@H](NC(=O)c1cnc(C)s1)C(=O)N[C@@H](COC)C(=O)N[C@@H](Cc1ccccc1)C(=O)[C@@]1(C)CO1]

### SUMMARY

This disclosure features pharmaceutical formulations (e.g., immediate release pharmaceutical formulations; e.g., solid dosage forms, e.g., tablets) that are useful for the oral administration of oprozomib, or a pharmaceutically acceptable salt thereof, to a human or animal subject as well as methods of making and using the formulations.

[I] Nausea, vomiting and diarrhea (“NVD”) is a gastrointestinal side effect that has been observed with oral administration of oprozomib, e.g., when oprozomib is formulated as a solution, a suspension, and in capsule and immediate release tablet forms. In-vivo animal studies have suggested that the NVD effect of oprozomib was the result of local proteasome inhibition, and that this local proteasome inhibition was not site (stomach or small intestine) specific. Rather, the NVD effect of oprozomib appeared to depend on the local concentration of oprozomib in the gastrointestinal (GI) tract (e.g., the stomach, duodenum, jejunum, ileum, and colon). Once oprozomib enters the lower GI tract, it is not absorbed well thus allowing oprozomib to cause GI tolerability issues. This implied that the occurrence of high local oprozomib concentrations in the stomach, duodenum and/or jejunum regions of the GI tract would likely trigger some degree of NVD in patients and potentially impact dose escalation. The present novel and inventive formulations, unknown and after much experimentation, aim to reduce or eliminate the NVD effect of oprozomib. As such, the novel and inventive formulations described herein can provide a reduced incidence or severity of one or more GI side effects (e.g., NVD).

Numerous other embodiments of the formulations of Tables I and Ia are set forth herein.

### Tables 1 and 1a

### Compositions of Oprozomib

TABLE 1

| | | | | | |
|---|---|---|---|---|---|
| | | 25 mg | 50 mg | | |
| | | Theoretical | Theoretical | | |
| | Percentage | Quantity | Quantity | | Reference to |
| Component | (% w/w) | (mg/unit) | (mg/unit) | Function | Standard |

Intra-Granular

| | | | | | |
|---|---|---|---|---|---|
| Oprozomib | 33.33 | 25.00 | 50.00 | Active | In-house |
| Microcrystalline | 30.84 | 23.13 | 46.26 | Filler | PhEur, |
| cellulose, PH 102 | | | | | USP/NF, JP |
| Lactose monohydrate | 30.83 | 23.12 | 46.25 | Filler | PhEur, |
| (Impalpable ® 313) | | | | | USP/NF, JP |
| Croscarmellose sodium | 4.0 | 3.000 | 6.00 | Disintegrant | PhEur, |
| | | | | | USP/NF, JP |
| Magnesium stearate | 0.50 | 0.375 | 0.750 | Lubricant | PhEur, |
| (non-bovine) | | | | | USP/NP, JP |

Extra-Granular

| | | | | | |
|---|---|---|---|---|---|
| Magnesium stearate | 0.50 | 0.375 | 0.750 | Lubricant | PhEur, |
| (non-bovine) | | | | | USP/NF, JP |
| Core Tablet Total | 100.00 | 75.00 | 150.0 | | |

Film Coating

| | | | | | |
|---|---|---|---|---|---|
| Opadry II ® White | 4.00 | 3.00 | 6.000 | Coating | Manufacturer |
| (85F18422) | | | | Material | Specification |
| Purified Water^{a} | — | — | — | Coating | USP |
| | | | | Solvent | |

^{a}Removed during the processing

TABLE 1a

| | | 25 mg | | |
|---|---|---|---|---|
| | | Theoretical | | |
| | Percentage | Quantity | | Reference to |
| Component | (% w/w) | (mg/unit) | Function | Standard |
| Oprozomib | 33.33 | 25.00 | Active | In-house |
| Silicon dioxide | 1.0 | 0.75 | Desiccant | USP/NF |
| Microcrystalline cellulose | 30.33 | 22.75 | Filler | PhEur, |
| | | | | USP/NF, JP |
| Lactose monohydrate | 30.34 | 22.76 | Filler | PhEur, |
| | | | | USP/NF, JP |
| Croscarmellose sodium | 4.0 | 3.000 | Disintegrant | PhEur, |
| | | | | USP/NF, JP |
| Magnesium stearate (non- | 0.50 | 0.375 | Lubricant | PhEur, |
| bovine) | | | | USP/NP, JP |
| Magnesium stearate (non- | 0.50 | 0.375 | Lubricant | PhEur, |
| bovine) | | | | USP/NF, JP |
| Core Tablet Total | 100.00 | 75.0 | | |
| Opadry II ® White | 4.00 | 3.00 | Coating | Manufacturer |
| (85F18422) | | | Material | Specification |
| Purified Water^{a} | — | — | Coating | USP |
| | | | Solvent | |

Opadry II® White (85F18422) is a well know coating material, commercially available from Colorcon, Inc, of West Point, Pa.

The formulations described in Tables 1 and 1a exhibited immediate release profiles, releasing equal or more than about 80% of the amount, or dose, of oprozomib, within about 30 minutes or longer (see FIG. 1 for Table 1 formulations and FIG. 12 for Table 1a formulations, respectively) under the dissolution conditions shown in Table 2.

TABLE 2

| | |
|---|---|
| Dissolution medium | About 0.01N Hydrochloric acid (HCl) |
| Media volume | About 900 milliliters (“mL”) |
| Temperature | 37 ± 0.5° C. |
| Apparatus | USP Apparatus 2 (Paddles) |
| Speed | 50 rpm through 60 minutes and 250 rpm for an |
| | additional 30 minutes |
| Sampling Time | About 15, 30, 45, 60, and 90 minutes |
| Sampling Volume | About 10 mL |

The extent of dissolution (USP apparatus 2) was followed and determined by UV detection (using a (UV/Vis spectrophotometer (Cary UV 50®)) with 10 mm flow cells of the Dissolution medium outlined in Table 2. The UV absorbance of the Dissolution medium was measured with six samples of about 10 mL each at a wavelength of 258 nm.

### [II]

The pharmaceutical formulations of oprozomib described herein provide an immediate release profile of oprozomib, e.g., conventional immediate release profiles, releasing at least or more than about 80% of the amount (or dose) of oprozomib at or about 30 minutes or longer, e.g., less than about 45 minutes, less than about 60 minutes, or less than about 90 minutes, as determined by UV under the following dissolution conditions:

| | |
|---|---|
| Dissolution medium | About 0.01N Hydrochloric acid (HCl) |
| Media volume | About 900 mL |
| Temperature | 37 ± 0.5° C. |
| Apparatus | USP Apparatus 2 (Paddles) |
| Speed | 50 rpm through 60 minutes and 250 rpm for an |
| | additional 30 minutes |
| Sampling Time | About 15, 30, 45, 60 and 90 minutes |
| Infinity Point | About 30 min after last sample |
| Sampling Volume | About 10 mL |

The immediate release pharmaceutical formulations of oprozomib described herein can provide one or more of the following advantages.

The immediate release pharmaceutical formulations of oprozomib described herein can provide therapeutically effective plasma exposure of oprozomib resulting in potent proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss). In some embodiments, the formulations described herein can deliver oprozomib with time to peak plasma concentrations of from about 15 to 180 minutes (e.g., from about 30 minutes to about 120 minutes; e.g., from about 30 minutes to about 60 minutes e.g., from about 30 minutes to about 45 minutes; e.g., about 30 minutes; e.g., about 45 minutes, e.g., about 60 minutes) as determined in dogs. As such, the formulations described herein can efficiently release oprozomib, e.g., to the stomach and proximal part of the small intestine, and do so over an immediate period of time and, in some instances, with improved bioavailability, pharmacokinetic (PK) and/or pharmacodynamic (PD) parameters, thereby increasing the likelihood that oprozomib will be absorbed by the duodenum and jejunum prior to excretion and/or degradation of oprozomib. In a preferred embodiment, the formulation increases the absorption of oprozomib in the duodenum and jejunum, leaving less of the drug to be present in the ileum and colon, which can cause tolerability issues. The present formulation can increase the GI tolerability of oprozomib, which can increase the likelihood of patient compliance with the dosage regimen. As such, the formulations described herein can provide a reduced incidence or severity of one or more GI side effects (e.g., NVD).

The immediate release pharmaceutical formulations of oprozomib described herein can be prepared in a form that is suitable for oral administration, which is among the preferred routes for administration of pharmaceuticals since this route is generally convenient and acceptable to patients. In certain embodiments, the formulations described herein can be orally administered as a solid dosage form (e.g., tablet, e.g., a matrix tablet; e.g., matrix pellets; e.g., particulates filled into capsule; e.g., self-emulsified drug delivery systems (SEDDS)).

### [III]

[A] Accordingly, in one aspect, this disclosure features immediate release pharmaceutical formulations, which include an effective amount of oprozomib, or a pharmaceutically acceptable salt thereof; in which equal or more than 80% of the amount or dose of oprozomib, or a pharmaceutically acceptable salt thereof, is released within about 90 minutes (e.g., less than about 30 minutes to less than about 90 minutes; e.g. less than about 90 minutes; e.g., less than about 30 to about 60 minutes; e.g., less than about 60 minutes; e.g., less than about 30 to about 60 minutes; e.g., about 30 minutes) as determined by UV under the following dissolution conditions:

| | |
|---|---|
| Dissolution medium | About 0.01N Hydrochloric acid (HCl) |
| Media volume | About 900 mL |
| Temperature | 37 ± 0.5° C. |
| Apparatus | USP Apparatus 2 (Paddles) |
| Speed | 50 rpm through 60 minutes and 250 rpm for an |
| | additional 30 minutes |
| Sampling Time | About 15, 30, 45, 60 and 90 minutes |
| Infinity Point | About 30 min after last sample |
| Sampling Volume | About 10 mL |

[B] In another aspect, this disclosure features immediate release pharmaceutical formulations, which include an effective amount of oprozomib, or a pharmaceutically acceptable salt thereof; in which the formulations provide a reduced incidence or severity of one or more side effects (e.g., NVD).

In certain embodiments, the formulations are in a form suitable for oral administration, e.g., a solid oral dosage form, e.g., a tablet, e.g., matrix tablet; e.g., matrix pellets; e.g., particulates filled into capsule; e.g., self-emulsified drug delivery systems (SEDDS).

[C] In a further aspect, this disclosure features immediate release pharmaceutical formulations, which include an effective amount of oprozomib, or a pharmaceutically acceptable salt thereof; in which the formulations provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss). In some embodiments, the formulations described herein can deliver oprozomib with time to peak plasma concentrations of about from about 15 to 180 minutes (e.g., from about 30 minutes to about 120 minutes; e.g., from about 30 minutes to about 60 minutes e.g., from about 30 minutes to about 45 minutes; e.g., about 30 minutes; e.g., about 45 minutes; e.g., about 60 minutes) as determined in dogs. As such, the formulations described herein can efficiently release oprozomib, e.g., to the stomach and proximal part of the small intestine, and do so over an immediate period of time and, in some instances, with improved bioavailability, pharmacokinetic (PK) and/or pharmacodynamic (PD) parameters, thereby increasing the likelihood that oprozomib will be absorbed by the duodenum and jejunum prior to excretion and/or degradation of oprozomib. In a preferred embodiment, the formulation increases the absorption of oprozomib in the duodenum and jejunum, leaving less of the drug to be present in the ileum and colon, which can cause GI tolerability issues. The formulations described herein can provide a reduced incidence or severity of one or more GI side effects (e.g., NVD). The present formulation can increase the GI tolerability of oprozomib, which can increase the likelihood of patient compliance with the dosage regimen, which can increase the likelihood of patient compliance with the dosage regimen.
[D] In one aspect, this disclosure features immediate release pharmaceutical formulations, which include an effective amount of oprozomib, or a pharmaceutically acceptable salt thereof; in which: (i) in which equal or more than about 80% of the amount or dose of oprozomib, or a pharmaceutically acceptable salt thereof, is released within about 90 minutes (e.g., at or about 30 minutes to less than about 90 minutes; e.g. less than about 90 minutes; e.g., at or about 30 to about 60 minutes; e.g., less than about 60 minutes; e.g., at or about 30 to about 45 minutes; e.g., at or about 30 minutes) as determined by UV under the following dissolution conditions:

| | |
|---|---|
| Dissolution medium | About 0.01N Hydrochloric acid (HCl) |
| Media volume | About 900 mL |
| Temperature | 37 ± 0.5° C. |
| Apparatus | USP Apparatus 2 (Paddles) |
| Speed | 50 rpm through 60 minutes and 250 rpm for an |
| | additional 30 minutes |
| Sampling Time | About 15, 30, 45, 60 and 90 minutes |
| Infinity Point | About 30 min after last sample |
| Sampling Volume | About 10 mL |

and

(ii) the formulations provide a reduced incidence or severity of one or more side effects (e.g., NVD).

In certain embodiments, the formulations are in a form suitable for oral administration, e.g., a solid oral dosage form, e.g., a solid oral dosage form, e.g., a tablet, e.g., matrix tablet; e.g., matrix pellets; e.g., particulates filled into capsule; e.g., self-emulsified drug delivery systems (SEDDS).

[E] In one aspect, this disclosure features extended release pharmaceutical formulations, which include an effective amount of oprozomib, or a pharmaceutically acceptable salt thereof; in which: (i) in which equal or more than about 80% of the amount or dose of oprozomib, or a pharmaceutically acceptable salt thereof, is released within about 90 minutes e.g. less than about 90 minutes; e.g., at or about 30 to about 60 minutes; e.g., at or about 60 minutes; e.g., at or about 30 to about 45 minutes; e.g., at or about 30 minutes) as determined by UV under the following dissolution conditions:

| | |
|---|---|
| Dissolution medium | About 0.01N Hydrochloric acid (HCl) |
| Media volume | About 900 mL |
| Temperature | 37 ± 0.5° C. |
| Apparatus | USP Apparatus 2 (Paddles) |
| Speed | 50 rpm through 60 minutes and 250 rpm for an |
| | additional 30 minutes |
| Sampling Time | About 15, 30, 45, 60 and 90 minutes |
| Infinity Point | About 30 min after last sample |
| Sampling Volume | About 10 mL |

(ii) the formulations provide a reduced incidence or severity of one or more side effects (e.g., NVD); and

(iii) the formulations provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss); in some embodiments, the formulations described herein can deliver oprozomib with time to peak plasma concentrations of about from about 15 to 180 minutes (e.g., from about 30 minutes to about 120 minutes; e.g., from about 30 minutes to about 60 minutes e.g., from about 30 minutes to about 45 minutes; e.g., about 30 minutes; e.g., 45 minutes; e.g., about 60 minutes) as determined in dogs; as such, the formulations described herein can efficiently release oprozomib, e.g., to the stomach and proximal part of the small intestine, and do so over an immediate period of time and, in some instances, with improved bioavailability, pharmacokinetic (PK) and/or pharmacodynamic (PD) parameters, thereby increasing the likelihood that oprozomib will be absorbed by the duodenum and jejunum prior to excretion and/or degradation of oprozomib. In a preferred embodiment, the formulation increases the absorption of oprozomib in the duodenum and jejunum, leaving less of the drug to be present in the ileum and colon, which can cause tolerability issues. The formulations described herein can provide a reduced incidence or severity of one or more GI side effects (e.g., NVD). The present formulation can increase the GI tolerability of oprozomib, which can increase the likelihood of patient compliance with the dosage regimen.

In certain embodiments, the formulations are in a form suitable for oral administration, e.g., a solid oral dosage form, e.g., a solid oral dosage form, e.g., a tablet, e.g., matrix tablet; e.g., matrix pellets; e.g., particulates filled into capsule; e.g., self-emulsified drug delivery systems (SEDDS).

An “effective amount” of oprozomib, or a pharmaceutically acceptable salt thereof, will vary from subject to subject, depending on species, age, and general condition of a subject, severity of the side effects or disorder, identity of the particular compound(s), mode of administration, and the like. As used herein, “an effective amount” refers to an amount of oprozomib, or a pharmaceutically acceptable salt thereof, that confers a therapeutic effect (e.g., controls, relieves, ameliorates, alleviates, or slows the progression of); or prevents (e.g., delays the onset of or reduces the risk of developing) a disease, disorder, or condition or symptoms thereof on the treated subject. The therapeutic effect may be objective (i.e., measurable by some test or marker) or subjective (i.e., subject gives an indication of or feels an effect).

[F] In one aspect, methods for treating cancer (e.g., multiple myeloma, e.g., multiple myeloma that is relapsed and/or refractory; e.g., Waldenström's macroglobulinemia; e.g., myelodysplastic syndromes; e.g., chronic lymphocytic leukemia; e.g., plasma cell leukemia; e.g., hepatocellular cancer; e.g., mantle cell leukemia) in a patient are featured, which include administering to the patient a formulation as described anywhere herein.

In another aspect, methods for treating autoimmune disease in a patient are featured, which include administering to the patient a formulation as described anywhere herein.

In another aspect, methods for treating graft or transplant-related condition in a patient are featured, which include administering to the patient a formulation as described anywhere herein.

In another aspect, methods for treating neurodegenerative disease in a patient are featured, which include administering to the patient a formulation as described anywhere herein.

In another aspect, methods for treating fibrotic-associated condition in a patient are featured, which include administering to the patient a formulation as described anywhere herein.

In another aspect, methods for treating ischemic-related condition in a patient are featured, which include administering to the patient a formulation as described anywhere herein.

In another aspect, methods for treating an infection in a patient are featured, which include administering to the patient a formulation as described anywhere herein.

In another aspect, methods for treating disease associated with bone loss in a patient are featured, which include administering to the patient a formulation as described anywhere herein.

[G] In one aspect, methods of preparing the formulations described herein are featured, which include granulating (i) oprozomib, or a pharmaceutically acceptable salt thereof; and (iii) one or more pharmaceutically acceptable excipients selected from one or more binders and one or more disintegrants in the presence of liquid comprising water.

In another aspect, formulations prepared by the methods described herein are featured e.g., by granulation, e.g., wet granulation (e.g., foam granulation, spray drying, lyophilization), direct compression, dry granulation (e.g., slugging, roller compaction), fluid bed granulation, emulsification, extrusion spheronization, hot melt extrusion, pelletization, drug layering, or coating.

[IV] Embodiments can include one or more of the following features.
The formulation can provide a reduced incidence or severity of one or more side effects (e.g., nausea/vomiting (NVD)).

The formulation can provide oprozomib with time to peak plasma concentrations of from about from about 15 to 180 minutes (e.g., from about 30 minutes to about 120 minutes; e.g., from about 30 minutes to about 60 minutes e.g., from about 30 minutes to about 45 minutes; e.g., about 30 minutes; e.g., about 45 minutes; e.g., about 60 minutes) as determined in dogs.

The formulation can be in a form that is suitable for oral administration.

The formulation can optionally include one or more pharmaceutically acceptable polymers.

In some embodiments, one or more pharmaceutically acceptable polymers is a matrix-forming polymer (e.g., a hydrophilic matrix-forming polymer, such as hydroxy propyl methylcellulose). In certain embodiments, the hydroxy propyl methylcellulose can have an apparent viscosity that is greater than 120 centipoise (“cP”) (2% water at 20° C.). For example, the hydroxy propyl methylcellulose can have an apparent viscosity of from 2500 cP (2% water at 20° C.) to 6000 cP (2% water at 20° C.). The formulation can include from 3.00 weight percent to 60.00 weight percent of the polymer (e.g., from 3.00 weight percent to 11.00 weight percent of the polymer; or from 13.00 weight percent to 22.00 weight percent of the polymer).

In one aspect of the invention, the formulation can include from about 5 weight percent to about 95 weight percent of oprozomib, or a pharmaceutically acceptable salt thereof (e.g., from about 10 weight percent to about 40 weight percent of oprozomib, or a pharmaceutically acceptable salt thereof, from about 15 weight percent to about 60 weight percent of oprozomib, or a pharmaceutically acceptable salt thereof, from about 20 weight percent to about 50 weight percent of oprozomib, or a pharmaceutically acceptable salt thereof; e.g., from about 25 weight percent to about 40 weight percent of oprozomib, or a pharmaceutically acceptable salt thereof, e.g., from about 25 weight percent to about 33 weight percent of oprozomib, or a pharmaceutically acceptable salt thereof, e.g., from about 30 weight percent to about 35 weight percent of oprozomib, or a pharmaceutically acceptable salt thereof, such as about 33.33 weight percent of oprozomib, or a pharmaceutically acceptable salt thereof; or about 15, 16, 17, 18, 19, 20, 21, 22, 23, 24, 25, 26, 27, 28, 29, 30, 31, 32, 33, 34, or 35 weight percent of oprozomib or a pharmaceutically acceptable salt thereof).

The formulation can include about 25 milligrams of oprozomib, or a pharmaceutically acceptable salt thereof; about 50 milligrams of oprozomib, or a pharmaceutically acceptable salt thereof; about 100 milligrams of oprozomib, or a pharmaceutically acceptable salt thereof; or about 400 milligrams of oprozomib, or a pharmaceutically acceptable salt thereof.

The formulation can include from about 20 weight percent to about 40 weight percent of oprozomib, or a pharmaceutically acceptable salt thereof, and about 25 milligrams of oprozomib, or a pharmaceutically acceptable salt thereof.

The formulation can include from about 20 weight percent to about 40 weight percent of oprozomib, or a pharmaceutically acceptable salt thereof, and about 50 milligrams of oprozomib, or a pharmaceutically acceptable salt thereof.

The formulation can include from about 20 weight percent to about 40 weight percent of oprozomib, or a pharmaceutically acceptable salt thereof, and about 100 milligrams of oprozomib, or a pharmaceutically acceptable salt thereof.

The formulation can include from about 25 weight percent to about 60 weight percent of oprozomib, or a pharmaceutically acceptable salt thereof, and about 400 milligrams of oprozomib, or a pharmaceutically acceptable salt thereof.

The oprozomib, or pharmaceutically acceptable salt thereof, can be a crystalline solid.

The oprozomib, or pharmaceutically acceptable salt thereof, can be an amorphous solid.

The formulation can further include one or more fillers. The one or more fillers can be selected from microcrystalline cellulose, lactose monohydrate, dibasic calcium phosphate (“DCP”), sucrose, glucose, mannitol, and sorbitol.

The formulation can optionally include one or more wetting agents (e.g., sodium laurel sulfate). Wetting agents can include surfactants or other surface active agents.

The formulation can further include one or more lubricants (e.g., magnesium stearate).

The formulation can further include one or more disintegrants (e.g., croscarmellose sodium).

The formulation can further include one or more coatings (e.g., Opadry II® White (85F18422)).

The formulation can include:

TABLE 3

| Component | Weight percent |
|---|---|
| Oprozomib, or a pharmaceutically acceptable | about 5 to about 95 |
| salt thereof | |
| One or more fillers | about 5 to about 95 |
| One or more disintegrants | about 0.01 to about 5 |
| One or more lubricants | about 0.01 to about 2 |
| One or more coatings | about 0.01 to about 10 |

For example, the formulation can include:

TABLE 4

| Component | Weight percent |
|---|---|
| Oprozomib, or a pharmaceutically acceptable | About 20 to about 40 |
| salt thereof | |
| One or more fillers | About 55 to about 70 |
| One or more disintegrants | About 3 to about 5 |
| One or more lubricants | About 0.6 to about 1.2 |
| One or more coatings | About 3 to about 5 |

In another aspect of the present invention, the formulation comprises:
i) from about 20 to about 35 weight percent oprozomib, or a pharmaceutically acceptable salt thereof;
ii) from about 30 to about 70 weight percent one or more fillers;
iii) from about 0.01 to about 2 weight percent one or more lubricants; and
iv) from about 3.5 to about 5 weight percent one or more disintegrants.

In another aspect, the present invention discloses a formulation comprising a desiccant.

The formulation can be a solid dosage form (e.g., a tablet).

The tablet can have a thickness of from about 2.5 millimeters to about 7 millimeters.

The tablet can have a thickness of from about 5 millimeters to about 6 millimeters.

The tablet can have a thickness of from about 3.0 millimeters to about 3.77 millimeters, e.g., about 3.04 millimeters; e.g., about 3.75 millimeters.

The tablet can have a hardness of from about 1.00 to about 25.00 kilopond (“kp”), e.g., about 5 kp. e.g., about 8 kp.

More than about 80% of the amount or dose of oprozomib, or a pharmaceutically acceptable salt thereof, is released within about 90 minutes.

More than about 80% of the amount or dose of oprozomib, or a pharmaceutically acceptable salt thereof, is released within about 60 minutes.

More than about 80% of the amount or dose of oprozomib, or a pharmaceutically acceptable salt thereof, is released at about 30 minutes.

In a certain cohort, a single dose of the formulation to a dog produces peak plasma concentration (Cₘₐₓ) of oprozomib of 66.4 ng/mL (having a standard deviation of 73.3) for a formulation comprising about 60 mg of oprozomib.

In a second cohort, the administration of the formulation (about 60 mg of oprozomib) to a dog produces an area under the concentration time curve to the last time point (AUC) of oprozomib of 28.6 ng*hr/mL, (having standard deviation of 18.6). The different results between the two cohorts may possibly be due to different metabolisms between the dogs.

The formulations can be stable upon actual or simulated storage under open conditions at 30° C./65% relative humidity (RH) for at least 1 month.

The formulations can be stable upon actual or simulated storage under open conditions at 40° C./75% RH for at least one month.

The formulations can be prepared by dry granulation, wet granulation or direct compaction.

The details of one or more embodiments of the invention are set forth in the accompanying drawings and the description below. Other features and advantages of the formulations and methods of making and using the same will be apparent from the description and drawings, and from the claims.

### DESCRIPTION OF DRAWINGS

FIG. 1 is a graph showing the release profile of the present formulation (see Table 1) that releases more than about 80% of oprozomib within about 30 minutes, within less than about 45 minutes, within less than about 60 minutes, within less than about 90 minutes, wherein the formulation storage time is T=0.

FIG. 2 shows an XRPD (X-ray powder diffraction) pattern of a crystalline form of oprozomib that is described in, e.g., U.S. Pat. No. 9,295,708.

FIG. 3 shows a DSC (differential scanning calorimetry) thermogram of a crystalline form of oprozomib that is described in, e.g., U.S. Pat. No. 9,295,708.

FIG. 4 shows a thermogravimetric (TG) thermogram of a crystalline form of oprozomib that is described in, e.g., U.S. Pat. No. 9,295,708.

FIG. 5 is a graph showing the release profile of the present formulation (see Table 1) that releases more than about 80% of the amount or dose of oprozomib within about 30 minutes, within less than about 45 minutes, within less than about 60 minutes, within less than about 90 minutes, and wherein the storage time is T=0.

FIG. 6 is a graph showing the release profile of the present formulation (see Table 1) that releases more than about 80% of the amount or dose of oprozomib within about 30 minutes, within less than about 45 minutes, within less than about 60 minutes, within less than about 90 minutes, and wherein the formulation was stored for 2 days (T=2) at 40° C./80% RH.

FIG. 7 shows the experimental design for the pharmacokinetic and pharmacodynamics (PK/PD) studies for the administration of the oprozomib formulations of Table 1 to dogs.

FIG. 8 shows pharmacokinetic (PK) data obtained for IR formulations of Table 1 and ER oprozomib formulations when administered to dogs.

FIG. 9 shows pharmacodynamic (PD) data obtained for IR formulations of Table 1, ER and oral suspension oprozomib formulations when administered to dogs.

FIG. 10 shows emesis events following oral administration of different oprozomib formulations of Table 1.

FIG. 11 shows the composition of the extended release (ER) formulation.

FIG. 12 is a graph showing the release profiles of the present formulation of Table 1a that releases more than about 80% of the amount or dose of oprozomib within about 30 minutes, within less than about 45 minutes, within less than about 60 minutes, within less than about 90 minutes, wherein the formulation storage time is T=0 (initial) and T=1 month at open conditions at 40° C./75% RH.

FIG. 13 is a graph showing the different dissolution profiles of experimental formulations comprising a desiccant different from the preferred desiccant wherein the formulation storage time is T=0 (Initial) and T=1 month at open conditions at 40° C./75% RH.

FIG. 14 is a manufacturing flow diagram for Oprozomib 25 mg, 50 mg and 200 mg IR Tablets for Table 1 Formulations.

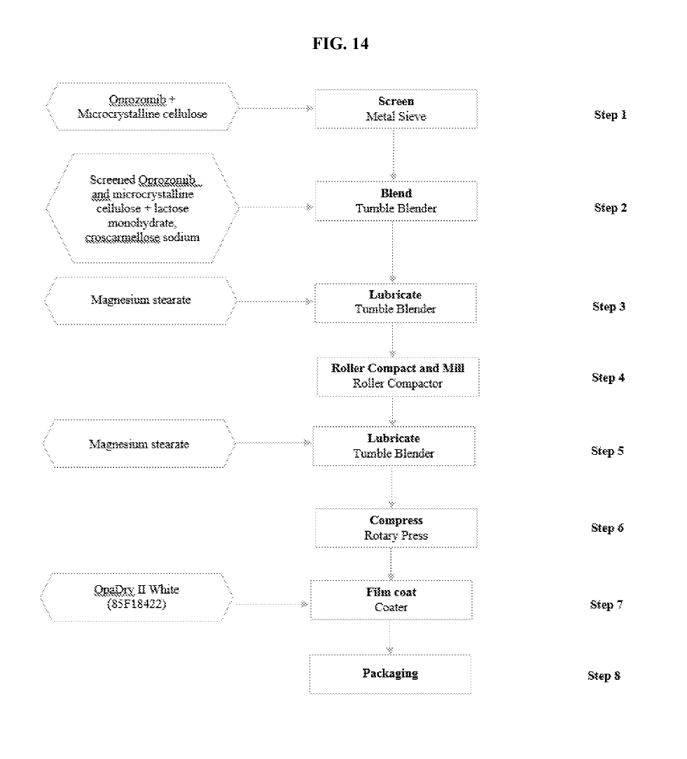

FIG. 15 is a manufacturing flow diagram for Oprozomib 25 mg IR Tablet for Table 1a Formulation.

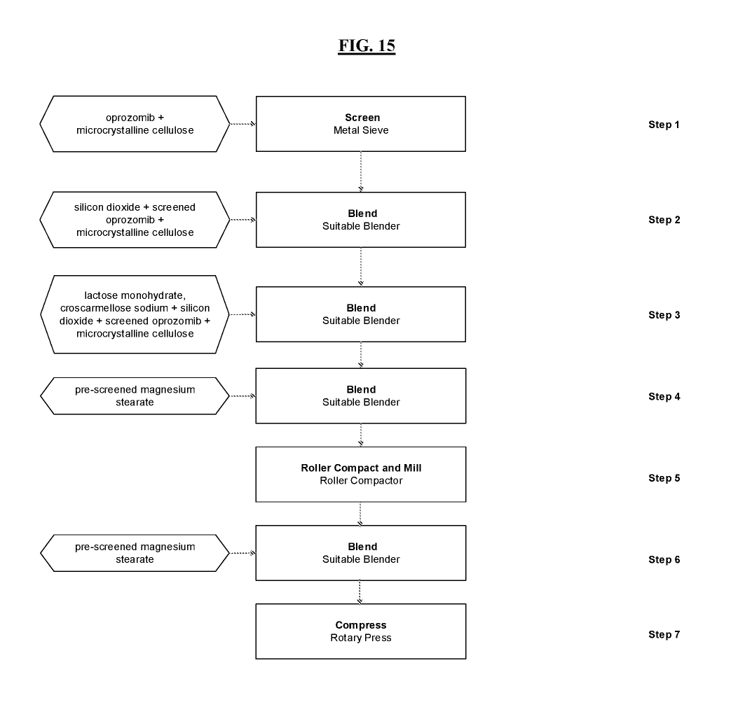

### DETAILED DESCRIPTION

This disclosure features immediate release pharmaceutical formulations (e.g., solid dosage forms, e.g., tablets) that are useful for the oral administration of oprozomib, or a pharmaceutically acceptable salt thereof, to a human or animal subject as well as methods of making and using the formulations.

The term “release” and “dissolution” are interchangeable terms in the present invention due to the formulation releasing or dissolving the pharmaceutical agent oprozomib to the patient or subject in an immediate fashion.

### [V] Formulation Components

[A] Typically, the formulations described herein include one or more components that modify the rate at which oprozomib is released from the formulation into the body. The one or more components can be present in the core of the formulation and/or in a coating(s) that surrounds the formulations.
[1] In some embodiments, the one or more components that modify the rate at which oprozomib is released from the formulation into the body can be one or more pharmaceutically acceptable polymers.

In some embodiments, the one or more pharmaceutically acceptable polymers can be any hydrophilic or lipophilic based controlled release polymers and excipients derived from natural, synthetic and/or semi-synthetic sources.

In certain embodiments, the one or more pharmaceutically acceptable polymers can be one or more matrix-forming polymers, e.g., one or more hydrophilic matrix-forming polymers.

In certain embodiments, the one or more pharmaceutically acceptable polymers can be a mixture of one or more matrix-forming polymers, e.g., one or more hydrophilic matrix-forming polymers, and one or more insoluble polymers, e.g., one or more ammoniomethacrylate copolymers.

In certain embodiments, the one or more hydrophilic matrix-forming polymers is hydroxy propyl methylcellulose (“HPMC”). In some embodiments, the one or more ammoniomethacrylate copolymer is Eudragit®.

In some embodiments, the formulations described herein can include one or more of the following:
Non-ionic soluble cellulose ethers, such as hydroxypropyl methylcellulose (HPMC, e.g., Methocel® K100LV, K4M, K15M, K100M; Benecel® MP843, MP 814, MP844; Metolose® 100, 4000, 15000 AND 100000 SR), hydroxypropyl cellulose (HPC, e.g., Klucel® GXF, MXF, HXF), hydroxyethyl cellulose (HEC, e.g., Natrosol® 250 HHX, HX, M, G) with various degrees of substitutions and viscosity grades
Nonionic homo-polymers of ethylene oxide such as polyethylene oxide (e.g. Polyox® WSR N-12K, WSR N-60K, WSR-301, WSR-coagulant, WSR-303, WSR-308)
Water-soluble natural gums of polysaccharides of natural origin, such as xanthan gum, alginate, and locust bean gum
Water swellable, but insoluble, high molecular weight homo-polymers and copolymers of acrylic acid chemically cross-linked with polyalkenyl alcohols with varying degree of cross-linking or particle size (Carbopol® 71G NF, 971P, 974P, 934P)
Polyvinyl acetate and povidone mixtures (Kollidon® SR)
Cross-linked high amylose starch
Ionic methacrylate copolymers (Eudragit® L30D, FS30D)
Fatty acids, fatty acid esters, mono-, di- and tri-glycerides of fatty acids, fatty alcohols, waxes of natural and synthetic origins with differing melting points e.g., stearic acid, lauryl, cetyl or cetostearyl alcohol, glyceryl behenate, carnauba wax, beeswax, candelila wax, microcrystalline wax and low molecular weight polyethylene
Insoluble polymers include ammoniomethacrylate copolymers (Eudragit® RL100, PO, RS100, PO, NE-30D, RL-30D, RS-30D, RL PO), ethyl cellulose (Ethocel®, Surelease®, Aquacoat® ECD), cellulose acetate (CA-398-10), cellulose acetate butyrate (CAB-381-20), cellulose acetate propionate (CAP-482-20), cellulose acetate phthalate (Aquacoat® CPD), polyvinylacetate (Kollicoat®)
Effervescent components include sodium bicarbonate, citric acid, stearic acid, and combinations thereof.

### [B] Oprozomib

[1] Oprozomib can be prepared, e.g., according to the synthetic route and procedures delineated in Example 1. As used herein, “oprozomib” without a modifier such as “in the form of a pharmaceutically acceptable salt” is intended to refer to the free-base form of oprozomib.
[2] In some embodiments, the formulations include oprozomib.

In some embodiments, the formulations include oprozomib in the form of a pharmaceutically acceptable salt.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.).

In certain embodiments, the formulations include amorphous oprozomib.

In certain embodiments, the formulations include one or more crystalline forms of oprozomib. An example of such a crystalline form of oprozomib is described in, e.g., US-2012-0077855, which is incorporated herein by reference in its entirety. Said crystalline form can include any one or more of the following features.

The crystalline form of oprozomib can have an X-ray powder diffraction pattern that includes one of the following characteristic peaks expressed in degrees 2θ: 9.4 (or about 9.4); 24.3 (or about 24.3); 11.1 (or about 11.1); or 15.3 (or about 15.3).

The crystalline form of oprozomib can have an X-ray powder diffraction pattern that includes any two, three or four of the following characteristic peaks: 9.4 (or about 9.4), 11.1 (or about 11.1), 15.3 (or about 15.3), and 24.3 (or about 24.3) (each expressed in degrees 2θ).

The crystalline form of oprozomib can have an X-ray powder diffraction pattern that includes the characteristic peak expressed in degrees 2θ at 9.4 (or about 9.4) and one of the following characteristic peaks: (i) the characteristic peak expressed in degrees 2θ at 24.3 (or about 24.3); or (ii) the characteristic peak expressed in degrees 2θ at 11.1 (or about 11.1); or (iii) the characteristic peak expressed in degrees 2θ at 15.3 (or about 15.3).

The crystalline form of oprozomib can have an X-ray powder diffraction pattern that includes the characteristic peaks expressed in degrees 2θ at 9.4 (or about 9.4), 11.1 (or about 11.1), and 24.3 (or about 24.3).

The crystalline form of oprozomib can have an X-ray powder diffraction pattern that includes the characteristic peaks expressed in degrees 2θ at 9.4 (or about 9.4), 11.1 (or about 11.1), 15.3 (or about 15.3), and 24.3 (or about 24.3).

The X-ray powder diffraction pattern of the crystalline form of oprozomib can also include one (or more) lower intensity characteristic peaks. The relative intensities of these additional peak(s) are, in general, lower than the relative intensities associated with the four characteristic peaks described above.

The crystalline form of oprozomib can have an X-ray powder diffraction pattern that includes characteristic peaks expressed in degrees 2θ at 9.4 (or about 9.4), 11.1 (or about 11.1), 15.3 (or about 15.3), 22.3 (or about 22.3), and 24.3 (or about 24.3).

The crystalline form of oprozomib can have an X-ray powder diffraction pattern that includes characteristic peaks expressed in degrees 2θ at 9.4 (or about 9.4), 11.1 (or about 11.1), 12.7 (or about 12.7), 15.3 (or about 15.3), 22.3 (or about 22.3), 24.3 (or about 24.3), and 28.3 (or about 28.3).

The crystalline form of oprozomib can have an X-ray powder diffraction pattern that includes characteristic peaks expressed in degrees 2θ at 9.4 (or about 9.4), 11.1 (or about 11.1), 12.7 (or about 12.7), 15.3 (or about 15.3), 20.9 (or about 20.9), 21.8 (or about 21.8), 22.3 (or about 22.3), 24.3 (or about 24.3), 28.3 (or about 28.3), 29.0 (or about 29.0), 29.7 (or about 29.7), and 30.5 (or about 30.5).

The crystalline form of oprozomib can have an X-ray powder diffraction pattern that includes characteristic peaks expressed in degrees 2θ at 8.9 (or about 8.9); 9.4 (or about 9.4); 9.8 (or about 9.8); 10.6 (or about 10.6); 11.1 (or about 11.1); 12.7 (or about 12.7); 15.3 (or about 15.3); 17.7 (or about 17.7); 19.0 (or about 19.0); 20.6 (or about 20.6); 20.9 (or about 20.9); 21.6 (or about 21.6); 21.8 (or about 21.8); 22.3 (or about 22.3); 22.8 (or about 22.8); 24.3 (or about 24.3); 24.7 (or about 24.7); 26.0 (or about 26.0); 26.4 (or about 26.4); 28.3 (or about 28.3); 29.0 (or about 29.0); 29.7 (or about 29.7); 30.2 (or about 30.2); 30.5 (or about 30.5); 30.8 (or about 30.8); 32.1 (or about 32.1); 33.7 (or about 33.7); 34.5 (or about 34.5); 35.1 (or about 35.1); 35.3 (or about 35.3); 37.9 (or about 37.9); and 38.5 (or about 38.5).

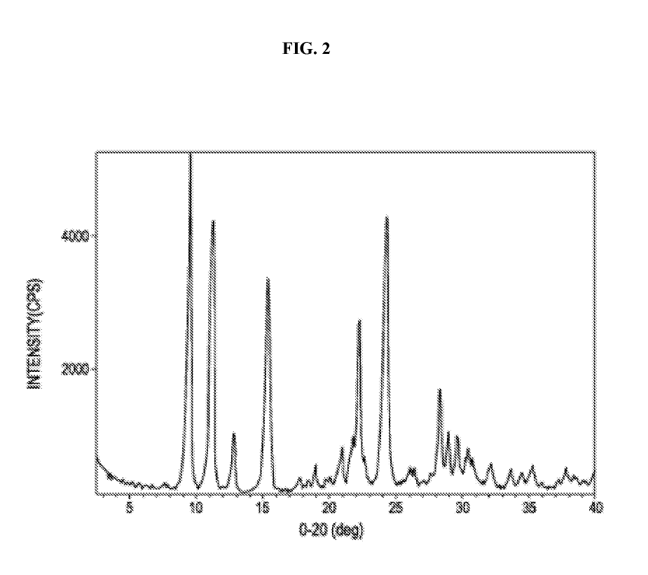

The crystalline form of oprozomib can have an X-ray powder diffraction pattern that is substantially the same as that shown (substantially as shown) in FIG. 2.

The term “about” when used in conjunction with defining a position of a characteristic peak in an X-ray powder diffraction pattern is intended to mean the stated degree 2θ value±0.2 degrees 2θ.

In some embodiments, the location(s) of characteristic peak(s) can be expressed to the nearest tenth (0.1) of a degree 2θ.

The crystalline form of oprozomib can also have one or more of the following characteristic features.

The crystalline form of oprozomib can have a differential scanning calorimetry pattern that includes a melting onset of about 140° C.

The crystalline form of oprozomib can have a differential scanning calorimetry pattern that includes a sharp endothermic maximum at about 147° C.

The crystalline form of oprozomib can have a differential scanning calorimetry pattern that includes a melting onset of about 140° C. and a sharp endothermic maximum at about 147° C.

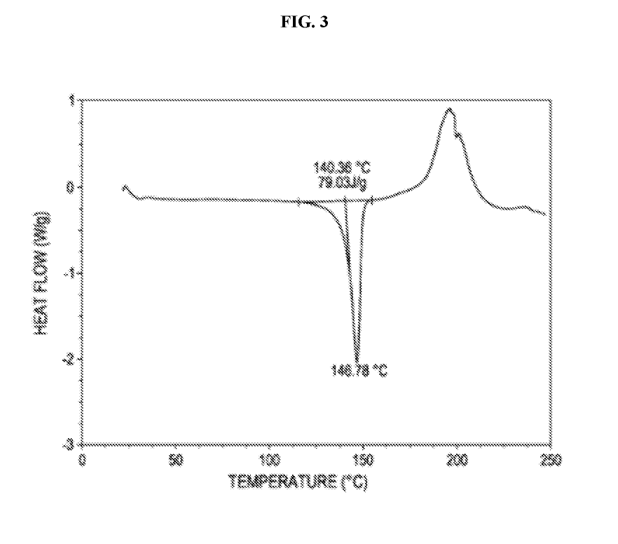

The crystalline form of oprozomib can have a differential scanning calorimetry pattern that is substantially the same as that shown (substantially as shown) in FIG. 3.

The crystalline form of oprozomib can have a melting point from about 140 to about 155° C. (e.g., from about 145 to about 150° C.).

The crystalline compound having Formula (II) can exhibit from 0.0 to 0.3% weight loss in the temperature range of 25 to 125° C.

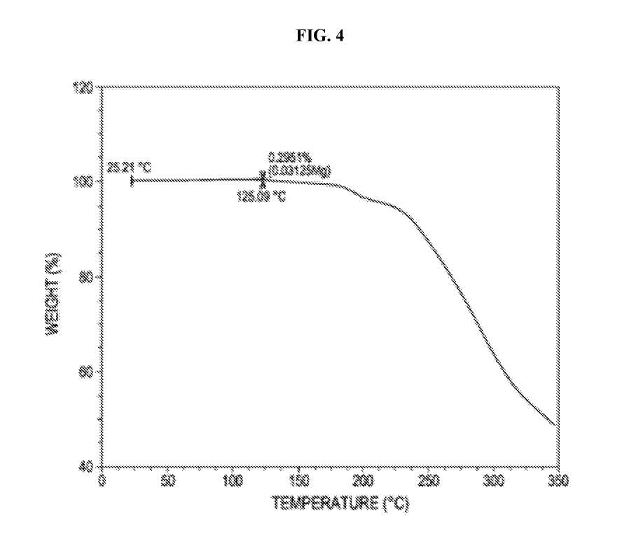

The crystalline form of oprozomib can have a thermogravimetric analysis pattern that is substantially the same as that shown (substantially as shown) in FIG. 4.

In certain embodiments, the formulations include both amorphous oprozomib and one or more crystalline forms of oprozomib as described anywhere herein.

In some embodiments, the formulations include oprozomib in the form of a pharmaceutically acceptable salt.

In certain embodiments, the formulations include amorphous oprozomib in the form of a pharmaceutically acceptable salt.

In certain embodiments, the formulations include one or more crystalline forms of oprozomib in the form of a pharmaceutically acceptable salt.

In some embodiments, the formulations include both oprozomib and oprozomib in the form of a pharmaceutically acceptable salt. These embodiments can include any combination of amorphous oprozomib, one or more crystalline forms of oprozomib, amorphous oprozomib in the form of a pharmaceutically acceptable salt, and one or more crystalline forms of oprozomib in the form of a pharmaceutically acceptable salt, each as described anywhere herein.

[3] The formulation can include from about 5 weight percent to about 95 weight percent of oprozomib, or a pharmaceutically acceptable salt thereof (e.g., from about 15 weight percent to about 60 weight percent of oprozomib, or a pharmaceutically acceptable salt thereof, from about 10 weight percent to about 40 weight percent of oprozomib, or a pharmaceutically acceptable salt thereof; from about 20 weight percent to about 50 weight percent of oprozomib, or a pharmaceutically acceptable salt thereof; e.g., from about 25 weight percent to about 40 weight percent of oprozomib, or a pharmaceutically acceptable salt thereof, from about 20 weight percent to about 33 weight percent of oprozomib, or a pharmaceutically acceptable salt thereof, e.g., from about 30 weight percent to about 35 weight percent of oprozomib, or a pharmaceutically acceptable salt thereof, such as about 33.33 weight percent of oprozomib, or a pharmaceutically acceptable salt thereof; or about 15, 16, 17, 18, 19, 20, 21, 22, 23, 24, 25, 26, 27, 28, 29, 30, 31, 32, 33, 34, or 35 weight percent of oprozomib or a pharmaceutically acceptable salt thereof).).

The formulation can include about 25 milligrams of oprozomib, or a pharmaceutically acceptable salt thereof; about 50 milligrams of oprozomib, or a pharmaceutically acceptable salt thereof; about 100 milligrams of oprozomib, or a pharmaceutically acceptable salt thereof; about 200 milligrams of oprozomib, or a pharmaceutically acceptable salt thereof or about 400 milligrams of oprozomib, or a pharmaceutically acceptable salt thereof.

In some embodiments, the formulations include from about 5 milligrams to about 500 milligrams (e.g., from about 5 milligrams to about 400 milligrams, from about 5.0 milligrams to about 250.0 milligrams, from about 25.0 milligrams to about 150.0 milligrams, from about 25.0 milligrams to about 130.0 milligrams, from about 25.0 milligrams to about 125.0 milligrams, from about 30.0 milligrams to about 70.0 milligrams, from about 55.0 milligrams to about 125.0 milligrams, from about 55.0 milligrams to about 65.0 milligrams, from about 80.0 milligrams to about 130.0 milligrams, from about 80.0 milligrams to about 120.0 milligrams, from about 85.0 milligrams to about 125.0 milligrams, from about 85.0 milligrams to about 95.0 milligrams, from about 115.0 milligrams to about 125.0 milligrams, from about 175.0 milligrams to about 225.0 milligrams) of oprozomib, or a pharmaceutically acceptable salt thereof.

In certain embodiments, the formulations can include from about 5.0 milligrams to about 250.0 milligrams (e.g., from about 25.0 milligrams to about 150.0 milligrams, from about 25.0 milligrams to about 130.0 milligrams, from about 25.0 milligrams to about 125.0 milligrams, from about 30.0 milligrams to about 70.0 milligrams, from about 35.0 milligrams to about 125.0 milligrams, from about 55.0 milligrams to about 65.0 milligrams, from about 80.0 milligrams to about 130.0 milligrams, from about 80.0 milligrams to about 120.0 milligrams, from about 85.0 milligrams to about 125.0 milligrams, from about 85.0 milligrams to about 95.0 milligrams, from about 115.0 milligrams to about 125.0 milligrams, from about 175.0 milligrams to about 225.0 milligrams) of oprozomib, or a pharmaceutically acceptable salt thereof. For example, the formulations can include about 50.00 milligrams of oprozomib, or a pharmaceutically acceptable salt thereof; about 60.00 milligrams of oprozomib, or a pharmaceutically acceptable salt thereof, about 90.00 milligrams of oprozomib, or a pharmaceutically acceptable salt thereof; about 100.00 milligrams of oprozomib, or a pharmaceutically acceptable salt thereof; about 120.00 milligrams of oprozomib, or a pharmaceutically acceptable salt thereof; or about 200.00 milligrams of oprozomib, or a pharmaceutically acceptable salt thereof.

In certain embodiments, the formulations include from about 20.0 milligrams to about 400.0 milligrams (e.g., from about 30.0 milligrams to about 300.0 milligrams, from about 55.0 milligrams to about 125.0 milligrams, from about 55.0 milligrams to about 65.0 milligrams, from about 80.0 milligrams to about 120.0 milligrams, from about 85.0 milligrams to about 125.0 milligrams, from about 85.0 milligrams to about 95.0 milligrams, from about 100.0 milligrams to about 125.0 milligrams) of oprozomib, or a pharmaceutically acceptable salt thereof. For example, the formulations can include about 20 milligrams of oprozomib, or a pharmaceutically acceptable salt thereof; about 25 milligrams of oprozomib, or a pharmaceutically acceptable salt thereof; about 30 milligrams of oprozomib, or a pharmaceutically acceptable salt thereof; about 35 milligrams of oprozomib, or a pharmaceutically acceptable salt thereof; about 40 milligrams of oprozomib, or a pharmaceutically acceptable salt thereof; about 45 milligrams of oprozomib, or a pharmaceutically acceptable salt thereof; about 50 milligrams of oprozomib, or a pharmaceutically acceptable salt thereof; about 55 milligrams of oprozomib, or a pharmaceutically acceptable salt thereof; about 100 milligrams of oprozomib, or a pharmaceutically acceptable salt thereof, or about 400 milligrams of oprozomib, or a pharmaceutically acceptable salt thereof.

In some embodiments, the formulations include from about 10 weight percent to about 50 weight percent (e.g., about 33.33 weight percent) of oprozomib, or a pharmaceutically acceptable salt thereof; and from about 10.0 milligrams to 400.0 milligrams (e.g., from about 25.0 milligrams to about 250.0 milligrams, from about 25.0 milligrams to about 125.0 milligrams, from about 30.0 milligrams to about 100.0 milligrams, from about 50.0 milligrams to about 125.0 milligrams, from about 55.0 milligrams to about 65.0 milligrams, from about 80.0 milligrams to about 130.0 milligrams, from about 80.0 milligrams to about 120.0 milligrams, from about 85.0 milligrams to about 125.0 milligrams, from about 85.0 milligrams to about 95.0 milligrams, from about 100.0 milligrams to about 125.0 milligrams, from about 175.0 milligrams to about 225.0 milligrams), e.g., about 25.00 milligrams, about 50.00 milligrams, about 60.00 milligrams, about 100.00 milligrams, about 150.00 milligrams, about 200.00 milligrams, about 250.00 milligrams, about 300.00 milligrams, or about 400.00 milligrams) of oprozomib, or a pharmaceutically acceptable salt thereof.

In some embodiments, the formulation can include from about 20.00 weight percent to about 40.00 weight percent of oprozomib, or a pharmaceutically acceptable salt thereof; and about 25.00 milligrams of oprozomib, or a pharmaceutically acceptable salt thereof, or from about 20.00 weight percent to about 40.00 weight percent of oprozomib, or a pharmaceutically acceptable salt thereof; and about 50.00 milligrams of oprozomib, or a pharmaceutically acceptable salt thereof.

In certain embodiments, the formulation can include from about 33.33 weight percent to about 45.00 weight percent of oprozomib, or a pharmaceutically acceptable salt thereof; and about 100.00 milligrams of oprozomib, or a pharmaceutically acceptable salt thereof.

[4] In some embodiments, any one or more of the features described throughout section [V][B][2] can be combined with any one or more of the features described throughout section [V][B][3].

### [C]

[1] In some embodiments, the formulations further include one or more pharmaceutically acceptable excipients. Pharmaceutically acceptable excipients include any and all fillers, binders, surfactants (wetting agents), disintegrants, desiccants, sugars, antioxidants, solubilizing or suspending agents, chelating agents, preservatives, colorants, buffering agents and/or lubricating agents, or combinations thereof, as suited to the particular dosage form desired and according to the judgment of the formulator. Remington's Pharmaceutical Sciences, Sixteenth Edition, E. W. Martin (Mack Publishing Co., Easton, Pa., 1980) discloses various pharmaceutically acceptable excipients used in preparing pharmaceutically acceptable formulations and known techniques for the preparation thereof. In general, the weight percent of the one or more pharmaceutically acceptable excipients (e.g., one or more fillers) varies with the weight percent and/or strength or purity of the oprozomib, or a pharmaceutically acceptable salt thereof; and, in some instances, the amount of oprozomib, or a pharmaceutically acceptable salt thereof, and the amount(s) of one or more other formulation components, e.g., a polymer component, e.g., HPMC.
[2] In some embodiments, the formulations include one or more fillers. As used herein, the term “filler” refers to a pharmaceutically acceptable substance that forms the bulk of a tablet when the amount of the oprozomib, or a pharmaceutically acceptable salt thereof (and, in some instances, the amount of oprozomib, or a pharmaceutically acceptable salt thereof, and the amount(s) of one or more other formulation components, e.g., a polymer component, e.g., HPMC) cannot provide this bulk (see The Theory and Practice of Industrial Pharmacy, Third Edition. Leon Lachman, Herbert Lieberman, and Joseph Kanig, editors. Lea & Febiger, Philadelphia. 1986, page 325).

Non-limiting examples of fillers include microcrystalline cellulose, lactose monohydrate, dibasic calcium phosphate (“DCP”), sucrose, glucose, mannitol, and sorbitol. Preferred fillers include microcrystalline cellulose and lactose monohydrate.

In some embodiments, the formulations can include two or more fillers. For example, the fillers can include microcrystalline cellulose (e.g., Avicel PH101 or Avicel PH102) and lactose monohydrate (e.g., Lactose 312 or Lactose 316).

In some embodiments, the formulations can include from about 5.00 weight percent to about 95.00 weight percent (e.g., from about 20.00 weight percent to about 80.00 weight percent, from about 30.00 weight percent to about 65.00 weight percent, from about 40.00 weight percent to about 65.00 weight percent, from about 60.00 weight percent to about 65.00 weight percent) of the one or more fillers. For example, the formulations can include about 30.84 weight percent of the one or more fillers or about 30.83 weight percent of the one or more fillers or about 61.67 weight percent of the one or more fillers.

In some embodiments, the weight percent ratio of the one or more fillers to the oprozomib, or a pharmaceutically acceptable salt thereof can be from about 0.5 to about 3.0. For example, the weight percent ratio of the one or more fillers to the oprozomib, or a pharmaceutically acceptable salt thereof can be about 0.9 to about 1.85.

[3] In some embodiments, the formulations include one or more disintegrants. As used herein, the term “disintegrant” refers to any pharmaceutically acceptable agent that is added to a pharmaceutical preparation to make it disintegrate (and thus release the active ingredient) on contact with water. Disintegrants are agents added to tablet (and some encapsulated) formulations to promote the breakup of the tablet (and capsule “slugs’) into smaller fragments in an aqueous environment thereby increasing the available surface area and promoting a more rapid release of the drug substance.

Non-limiting examples of disintegrants include starch (e.g., corn starch or potato starch), Pregelatinized Starch (Starch 1500), Microcrystalline Cellulose, Modified (crosslinked) Starches (e.g.; Sodium Carboxymethyl Starch), Cross-linked polyvinylpyrrolidone, Modified (crosslinked) Cellulose

(i.e. Ac-Di-Sol (Accelerates Dissolution), croscarmellose sodium, Nymcel), crosslinked alginic acid (such as Alginic acid NF and Satiaigin®), natural super disintegrants (such as soy polysaccharides or Emcosoy®), and calcium silicate. An exemplary disintegrant is croscarmellose sodium.

In some embodiments, the formulations include from about 0.50 weight percent to about 5.00 weight percent (e.g., from about 3.0 weight percent to about 4.50 weight percent, from about 3.50 weight percent to about 4.25 weight percent, e.g., about 4.00 weight percent) of the one or more disintegrants.

[4] In some embodiments, the formulations include one or more lubricants. As used herein, the term “lubricant” refers to a pharmaceutically acceptable substance that reduces the friction associated with tablet ejection between the walls of the tablet and the walls of a cavity used to form the tablet (see The Theory and Practice of Industrial Pharmacy, Third Edition. Leon Lachman, Herbert Lieberman, and Joseph Kanig, editors. Lea & Febiger, Philadelphia. 1986, page 328).

Suitable lubricants include magnesium stearate; metal stearates, glyceryl behenate, sodium stearyl fumarate, hydrogenated vegetable oils, or fatty acids. An exemplary lubricant is magnesium stearate.

In some embodiments, the formulations can include from about 0.10 weight percent to about 2.00 weight percent (e.g., from 0.50 weight percent to about 1.20 weight percent, e.g., about 1.0 weight percent) of a lubricant.

In some embodiments, the formulations include materials, which are both lubricated (can function as a lubricant) and can function as a filler (e.g., siliconized microcrystalline cellulose (MCC)). These materials can be present in amounts as described above and/or in section [V][C][2].

In some embodiments, viscosity control agents (e.g., suspending agents), which may be polymeric or colloidal (e.g., modified cellulose polymers such as methylcellulose, hydoxyethylcellulose, hydrophobically modified hydroxyethylcellulose, and cross-linked acrylate polymers such as Carbomer, hydrophobically modified polyethers) can be included in the composition, in the core or wall.

In some embodiments, desiccants, such as silicas, either hydrophobic or hydrophilic, can be included at a concentration from 0.01 to 20%, more preferable from 0.5 to 5%, and most preferably from 1 to 2% by the weight of the capsule composition. Examples of hydrophobic silicas include silanols, surfaces of which are treated with halogen silanes, alkoxysilanes, silazanes, and siloxanes, such as SIPERNAT D17, AEROSIL, R972 and R974 available from Degussa, Exemplary hydrophilic silicas are AEROSIL 200, SIPERNAT 22S, SIPERNAT 50S (available from Degussa), and SYLOID 244 (available from W.R. Grace).

In some embodiments, the formulations include one or more desiccants (Table 1a; e.g., silicon dioxide). Preferably, the desiccant is silicon dioxide, more preferably porous silicon dioxide, more preferably silicon dioxide having a surface area of about 500 to about 1000 m²/g, most preferably having a surface area of about 700 m²/g. In one aspect of the invention, the silicon dioxide has an average pore volume of about 0.2 to about 1 cc/g, most preferably about 0.4 cc/g. SYLOID® 63FP, a porous silicon dioxide commercially available from W.R. Grace, Columbia, Md., was used in the formulation in Table 1a. Various grades of silicon dioxide, such as Cab-O-Sil M-5P®, commercially available from Cabot Corporation, Boston, Mass., were investigated but did not provide satisfactory dissolution profiles after storage at 40° C./75% RH under open conditions for 1 month.

In some embodiments, such as when the formulation is comprised of a tablet or capsule form, the formulation can further include one or more coatings (e.g., Opadry II® White (85F18422) or another color).

[5] In some embodiments, any one or more of the features described throughout section [V][C][1] can be combined with any one or more of the features described throughout sections [V][C][2], or [V][C][3], or [V][C][4].

In some embodiments, any one or more of the features described throughout section [V][C][1] can be combined with any one or more of the features described throughout sections [V][C][2] and [V][C][3] or [V][C][4].

In some embodiments, any one or more of the features described throughout section [V][C][1] can be combined with any one or more of the features described throughout sections [V][C][2], [V][C][3] and [V][C][4].

### [D] Non-limiting Combinations of Formulation Components

[1]

In some embodiments, the formulations include:

(i) oprozomib, or a pharmaceutically acceptable salt thereof; and

(ii) one or more components that modify the rate at which oprozomib is released from the formulation into the body (e.g., one or more agents).

In certain embodiments, the formulations described above can include any one or more of the features described throughout sections [V][B][2] and/or [V][B][3] and/or [V][B][4].

In certain embodiments, the formulations described above can include:

(i) any one or more of the features described throughout sections [V][A][1]; and/or

(ii) any one or more of the features described throughout sections [V][B][2] and/or [V][B][3] and/or [V][B][4].

[2] In some embodiments, the formulations described above include:

(i) oprozomib, or a pharmaceutically acceptable salt thereof;

(ii) optionally one or more components that modify the rate at which oprozomib is released from the formulation into the body (e.g., one or more agents and mixtures thereof); and

(iii) one or more pharmaceutically acceptable excipients (e.g., one or more fillers and/or one or more disintegrants and/or one or more lubricants).

For example, the formulations described above can include components in Tables 5 and 5a:

TABLE 5

| Component | Weight percent |
|---|---|
| Oprozomib, or a | About 15 to about 70 (e.g., about 15 to about |
| pharmaceutically | 60, about 15 to about 60, e.g., about 20 to about |
| acceptable salt | 50, e.g., about 25 to about 40, e.g., about 30 to |
| thereof | about 35, e.g., about 33.) |
| One or more fillers | About 30 to about 80 (e.g., from about 30 to |
| | about 70, e.g., about 40 to about 65, e.g., about |
| | 50 to about 65, e.g., about 60 to about 65, e.g., |
| | about 30, e.g., about 30.83, e.g., about 61.67) |
| One or more | About 0.01 to about 5 (e.g., about 3 to about |
| disintegrants | 4.5, e.g., about 3.5 to about 4.25, e.g., about 4) |
| One or more lubricants | About 0.01 to about 2 weight percent (e.g., |
| | about 0.5 to about 5, e.g., about 1 weight |
| | percent) |
| One or more coatings | About 0.01 to about 10 (e.g., about 3 to about |
| | 5, e,g., about 4) |

TABLE 5a

| Component | Weight percent |
|---|---|
| Oprozomib, or a | About 15 to about 70 (e.g., about 15 to about |
| pharmaceutically | 60, about 15 to about 60, e.g., about 20 to about |
| acceptable salt | 50, e.g., about 25 to about 40, e.g., about 30 to |
| thereof | about 35, e.g., about 33.) |
| One or more | About 0.1 to about 2 |
| desiccants | |
| One or more fillers | About 30 to about 80 (e.g., from about 30 to |
| | about 70, e.g., about 4 to about 65, e.g., about |
| | 50 to about 65, e.g., about 60 to about 65, e.g., |
| | about 30, e.g., about 30.83, e.g., about 61.67) |
| One or more | About 0.01 to about 5 (e.g., about 3 to about |
| disintegrants | 4.5, e.g., about 3.5 to about 4.25, e.g., about 4) |
| One or more lubricants | About 0.01 to about 2 weight percent (e.g., |
| | about 0.5 to about 5, e.g., about 1.0 weight |
| | percent) |
| One or more coatings | About 0.01 to about 10 (e.g., about 3 to about 5, |
| | e,g., about 4) |

In certain embodiments, the formulations described above can include any one or more of the features described throughout sections [V][A][1].

In certain embodiments, the formulations described above can include any one or more of the features described throughout sections [V][B][2] and/or [V][B][3] and/or [V][B][4].

In certain embodiments, the formulations described above can include any one or more of the features described throughout sections [V][C][1] and/or [V][C][2] and/or [V][C][3] and/or [V][C][4] and/or [V][C][5].

In certain embodiments, the formulations described above can include:

(i) any one or more of the features described throughout sections [V][A][1];

(ii) any one or more of the features described throughout sections [V][B][2] and/or [V][B][3] and/or [V][B][4]; and

(iii) any one or more of the features described throughout sections [V][C][1] and/or [V][C][2] and/or [V][C][3] and/or [V][C][4] and/or [V][C][5].

In some embodiments, any one or more of the features described throughout section [V] above can be combined with any one or more of the features described throughout sections [III] and/or [IV] above.

### [VI] Dosage Forms

In general, oral administration of the formulations is preferred, and the formulations can be in any form that is suitable for oral administration (e.g., any conventional oral dosage forms including, but not limited to, solid dosage forms such as a tablet, a pill, a hard or soft capsule, a dragee, a lozenge, a cachet, a sachet, a powder (e.g., dispensable powder), granules; and liquid preparations such as syrups, slurries, gels, pellets, particulates, elixirs, emulsions and aqueous suspensions, dispersions, solutions, and concentrated drops, or any other form reasonably adapted for oral administration).

In some embodiments, the formulations can be in the form of a discrete, solid oral dosage unit (e.g. a capsule, a tablet, or a dragee) containing a predetermined amount of any one or more of the components described herein, e.g., as described throughout section [V].

In some embodiments, the formulations can be in the form of a tablet. Such forms can be shaped and dimensioned as desired. In certain embodiments, the formulations can be in the form of a tablet that is capsule-shaped. In some embodiments, the tablet can be a modified capsule shaped or modified oval shaped core tablet. In certain embodiments, the formulations can be in the form of a tablet having a thickness of from about 2.5 to about 12.0 millimeters (mm) (e.g., from about 2.0 to about 4.0 millimeters, from about 3.0 to about 3.8 millimeters, from about 3.0 millimeters to about 3.77 millimeters, or from about 5 to 7 mm).

In certain embodiments, the formulations can be in the form of a “compressed tablet,” which as used herein refers to a plain, uncoated tablet for oral ingestion. Compressed tablet are typically prepared by a single compression or by pre-compaction tapping followed by a final compression (e.g., using a Carver press, rotary press, single station tablet press). The tablets can be scored, printed, and/or debossed or embossed with desired identifier markings. In some embodiments, the tablets can have a hardness of from about 3.0 kp to about 14.0 kp (e.g., from about 3.0 kp to about 12.0 kp, from about 5.0 kp to 10.0 kp, from about 5.0 kp to about 8.0 kp).

In certain embodiments, the tablet can be a coated tablet. As a further example, tablets can also be coated with a conventional coating material such as Opadry™ White 85F18422 (or another color). In some embodiments, the coating is present from about 1.00 to about 5.00 weight percent of the tablet. For example, the coating can be present at about 4.00 weight percent.

In certain embodiments, the weight of the tablet can be from about 5 milligrams to about 1,500 milligrams (e.g., about 60 milligrams to about 1,000 milligrams; from about 70 milligrams to about 500 milligrams; e.g., about 75 milligrams, about 78 milligrams, about 80 milligrams, about 100 milligrams, about 150 milligrams, about 156 milligrams, about 200 milligrams, about 250 milligrams, about 400 milligrams or about 450 milligrams).

In general, the formulations can be prepared by any suitable and conventional method of pharmacy known in the art, which includes the step of bringing into association any one or more of the components described herein, e.g., as described throughout section [V]. Methods of preparation can include one or a combination of methods including: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986).

In some embodiments, the formulations can be obtained, for example, by performing one or more of the following steps: (i) combining (e.g., uniformly and intimately admixing so as to disperse the active ingredient evenly throughout the composition, e.g., to facilitate subdivision of the formulation into unit dosage forms) the active ingredient, surfactant(s), and any other component(s) described herein to provide a mixture; (ii) screening, sieving, grinding, and/or milling the resulting mixture; (iii) processing the mixture of granules, after adding suitable auxiliaries, if desired; (iv) shaping and optionally coating the product to obtain tablets or dragee cores; or (v) adding the processed formulation to a vessel suitable for oral administration, such as a capsule.

In certain embodiments, the formulations can be prepared using wet granulation techniques known in the art, which can include the steps of milling and sieving of the ingredients, dry powder mixing, wet massing, granulation and final grinding. In some embodiments, the wet granulation techniques such as high shear granulation, fluid bed granulation, extrusion spheronization etc. can better accommodate the micronized active ingredients and can result in formulations having enhanced powder flow (for encapsulation) and dissolution properties.

In certain embodiments, the formulations can be prepared using dry granulation techniques known in the art, which can include the steps of milling and sieving of the ingredients, dry powder mixing, and final blending.

In certain embodiments, compressed tablets can be prepared by compressing, in a suitable machine, such as a roller compaction machine, the formulation in a free-flowing form, such as a powder or granules. Molded tablets can be made by molding, in a suitable machine, the powdered formulation moistened with an inert liquid diluent.

The Examples section provides more specific methods for preparing the formulations described herein.

### [VII] Non-Limiting Properties of Formulations

The formulations described herein can have any one or more of the following properties.

[A] In some embodiments, more than about 80% of the amount or dose of oprozomib, or a pharmaceutically acceptable salt thereof, is released about 30 minutes or longer, e.g., about 30 minutes, within about 45 minutes, or within about 60 minutes.

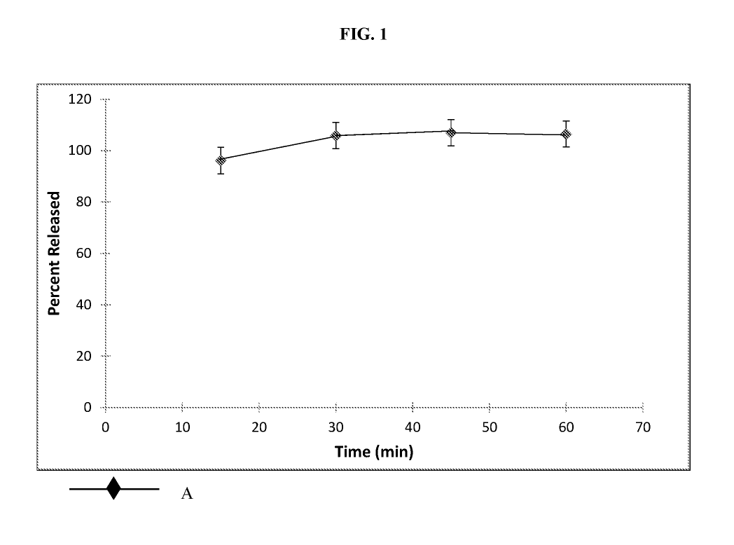

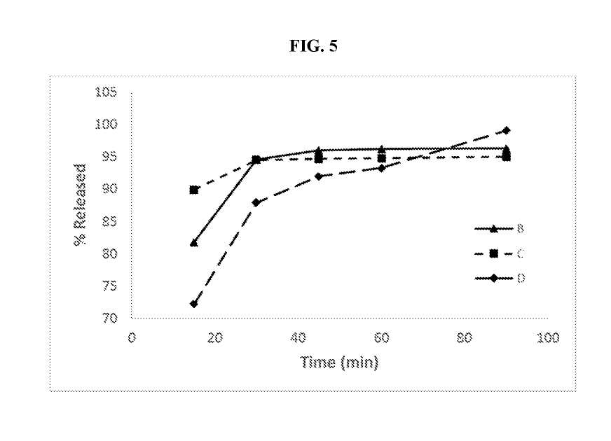

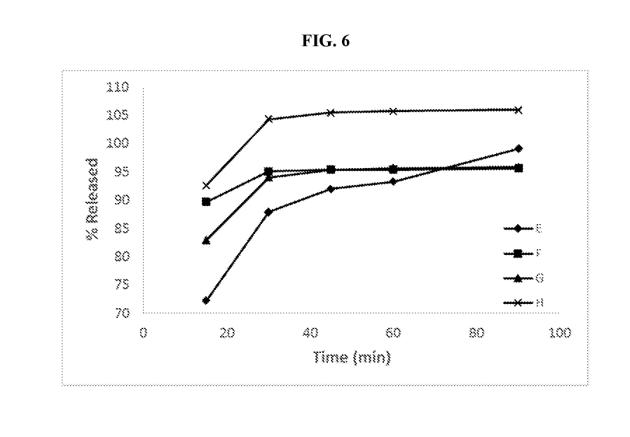

In some embodiments, the formulations can exhibit any one, two, three, four, five, six, seven and/or eight of the release profile properties delineated in Table 6 below. See FIG. 1 for (A); see FIG. 5 for (B), (C), and (D); and see FIG. 6 for (E), (F), (G), and (H).

TABLE 6
Dissolution Profile for Oprozomib Immediate
Release 25-mg Tablets of Table 1

| Storage time/ | | T = 2 days |
|---|---|---|
| conditions | T = 0 | (40 C./80% RH) |
| | (A) Average (Avg) % | |
| Time (min) | oprozomib released | (E) Avg % released* |
| 15 | 96.2 | 92.6 |
| 30 | 105.9 | 104.3 |
| 45 | 107 | 105.5 |
| 60 | 106.5 | 105.7 |
| Time (min) | (B) Avg % released | (F) Avg % released* |
| 15 | 80.1 | 72.2 |
| 30 | 95.1 | 87.9 |
| 45 | 97.9 | 92 |
| 60 | 98.5 | 93.3 |
| 90 | 100.4 | 99.1 |
| Time (min) | (C) Avg % released* | (G) Avg % released* |
| 15 | 89.9 | 89.7 |
| 30 | 94.5 | 95.1 |
| 45 | 94.7 | 95.4 |
| 60 | 94.8 | 95.4 |
| 90 | 95 | 95.6 |
| Time (min) | (D) Avg % released* | (H) Avg % released* |
| 15 | 81.8 | 82.9 |
| 30 | 94.6 | 94 |
| 45 | 96 | 95.4 |
| 60 | 96.2 | 95.6 |
| 90 | 96.3 | 95.8 |

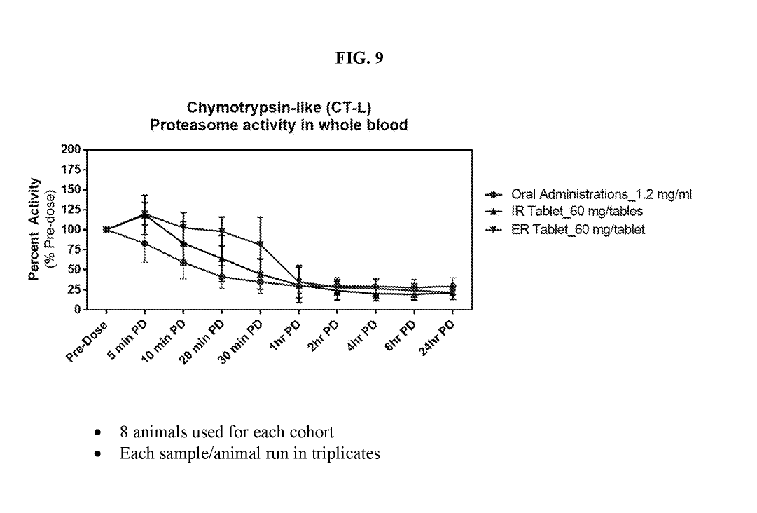

*6 tablets

[B] In some embodiments, the formulations provide a reduced incidence or severity of one or more side effects (e.g., NVD).
[C] In some embodiments, the formulations provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss). In some embodiments, the formulations described herein can deliver oprozomib with time to peak plasma concentrations of from about 30 to about 180 minutes (e.g., from about 30 minutes to about 60 minutes; e.g., from about 30 minutes to about 45 minutes; e.g., from about 45 minutes to about 60 minutes; e.g., about 30 minutes; e.g., about 45 minutes; e.g., about 60 minutes) (See FIG. 9) as determined in dogs; as such, the formulations described herein can efficiently release oprozomib, e.g., to the stomach and proximal part of the small intestine, and do so over an immediate period of time and, in some instances, with improved bioavailability, pharmacokinetic (PK) and/or pharmacodynamic (PD) parameters, thereby increasing the likelihood that oprozomib will be absorbed by the duodenum and jejunum prior to excretion and/or degradation of oprozomib. In a preferred embodiment, the formulation increases the absorption of oprozomib in the duodenum and jejunum, leaving less of the drug to be present in the ileum and colon, which can cause tolerability issues. The present formulation can increase the GI tolerability of oprozomib, which can increase the likelihood of patient compliance with the dosage regimen, which can increase the likelihood of patient compliance with the dosage regimen.

In certain embodiments, a single dose of the formulation to a dog produces peak plasma concentration (Cₘₐₓ) of oprozomib of 66.4 ng/mL (having a standard deviation of 73.3) for a formulation comprising about 60 mg of oprozomib.

In certain embodiments, the administration of the formulation (about 60 mg of oprozomib) to a dog produces an area under the concentration time curve to the last time point (AUC) of oprozomib of 28.6 ng*hr/mL, having a standard deviation of 18.6).

[D] The present formulations may be packaged in bottles, bottles with caps, bottles with desiccants, blister packs or other well accepted packaging in the industry.

In some embodiments, the 25 mg and 50 mg formulations are stable upon actual or simulated storage in open conditions at 25° C./60% relative humidity for at least 1 month.

Stability studies were carried out using one of the following procedures:

(A) Tablets were packaged in 75 cc white HDPE bottles with closures and stored at 30° C.±2° C./65% relative humidity (RH)±5% RH and 40° C.±2° C./75% RH±5% RH. Tablets were tested for appearance, hardness, assay and impurities and dissolution at pre-determined time points.
(B) Tablets were packaged in 75 cc white HDPE bottles with closures and desiccant and stored at 25° C.±2° C./60% relative humidity (RH)±5% RH and 40° C.±2° C./75% RH±5% RH. Tablets were tested for appearance, hardness, assay and impurities and dissolution at pre-determined time points. In preferred embodiments, when the formulation is stored in a 75 cc white HDPE bottle with desiccant at 25° C./60% relative humidity for at least 1 month, the formulation shows less than about 1.0% degradation of oprozomib. In more preferred embodiments, the amount of degradation of oprozomib is less than 0.5%, 0.4%, 0.3%, 0.2%, and in some instances, less than 0.1%.
(C) In particular, impurities PR-059176 (PR-176) and PR-487 were detected and measured.

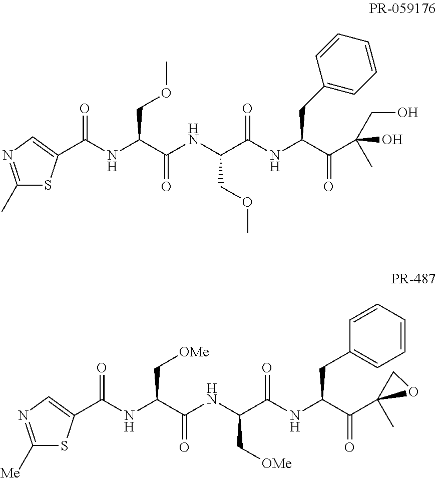 [SMILES: COC[C@H](NC(=O)c1cnc(C)s1)C(=O)N[C@@H](COC)C(=O)N[C@@H](Cc1ccccc1)C(=O)[C@](C)(O)CO ; COC[C@H](NC(=O)c1cnc(C)s1)C(=O)N[C@H](COC)C(=O)N[C@@H](Cc1ccccc1)C(=O)[C@@]1(C)CO1]

### [VIII] Uses of Formulations

Orderly protein degradation is crucial to the maintenance of normal cell functions, and the proteasome is integral to the protein degradation process. The proteasome controls the levels of proteins that are important for cell-cycle progression and apoptosis in normal and malignant cells; for example, cyclins, caspases, BCL2 and NF-kB (Kumatori et al., Proc. Natl. Acad. Sci. USA (1990) 87:7071-7075; Almond et al., Leukemia (2002) 16: 433-443). Thus, it is not surprising that inhibiting proteasome activity can translate into therapies to treat various disease states, such as malignant, non-malignant and autoimmune diseases, depending on the cells involved.

Both in vitro and in vivo models have shown that malignant cells, in general, are susceptible to proteasome inhibition. In fact, proteasome inhibition has already been validated as a therapeutic strategy for the treatment of multiple myeloma. This could be due, in part, to the highly proliferative malignant cell's dependency on the proteasome system to rapidly remove proteins (Rolfe et al., J. Mol. Med. (1997) 75:5-17; Adams, Nature (2004) 4: 349-360). Therefore, certain embodiments of the invention relate to a method of treating a cancer, comprising administering to a subject in need of such treatment an effective amount of a proteasome inhibitor compound disclosed herein. As used herein, the term “cancer” includes, but is not limited to, blood borne and solid tumors. Cancer refers to disease of blood, bone, organs, skin tissue and the vascular system, including, but not limited to, cancers of the bladder, blood, bone, brain, breast, cervix, chest, colon, endometrium, esophagus, eye, head, kidney, liver, lung, lymph nodes, mouth, neck, ovaries, pancreas, prostate, rectum, renal, skin, stomach, testis, throat, and uterus. Specific cancers include, but are not limited to, leukemia (acute lymphocytic leukemia (ALL), acute myelogenous leukemia (AML), chronic lymphocytic leukemia (CLL), chronic myelogenous leukemia (CML), hairy cell leukemia), mature B cell neoplasms (small lymphocytic lymphoma, B cell prolymphocytic leukemia, lymphoplasmacytic lymphoma (such as Waldenström's macroglobulinemia or indolent lymphoma), splenic marginal zone lymphoma, plasma cell myeloma, plasma cell leukemia, plasmacytoma, monoclonal immunoglobulin deposition diseases, heavy chain diseases, extranodal marginal zone B cell lymphoma (MALT lymphoma), nodal marginal zone B cell lymphoma (NMZL), a gastrointestinal tumor (e.g., a gastrointestinal stromal tumor (GIST)), follicular lymphoma, mantle cell lymphoma/leukemia, diffuse B cell lymphoma, mediastinal (thymic) large B cell lymphoma, intravascular large B cell lymphoma, primary effusion lymphoma and Burkitt lymphoma/leukemia), mature T cell and natural killer (NK) cell neoplasms (T cell prolymphocytic leukemia, T cell large granular lymphocytic leukemia, aggressive NK cell leukemia, adult T cell leukemia/lymphoma, extranodal NK/T cell lymphoma, enteropathy-type T cell lymphoma, hepatosplenic T cell lymphoma, blastic NK cell lymphoma, mycosis fungoides (Sezary syndrome), primary cutaneous anaplastic large cell lymphoma, lymphomatoid papulosis, angioimmunoblastic T cell lymphoma, unspecified peripheral T cell lymphoma and anaplastic large cell lymphoma), Hodgkin's lymphoma (nodular sclerosis, mixed celluarity, lymphocyte-rich, lymphocyte depleted or not depleted, nodular lymphocyte-predominant), myeloma (multiple myeloma, indolent myeloma, smoldering myeloma), chronic myeloproliferative disease, myelodysplastic/myeloproliferative disease, myelodysplastic syndromes, immunodeficiency-associated lymphoproliferative disorders, histiocytic and dendritic cell neoplasms, mastocytosis, chondrosarcoma, Ewing sarcoma, fibrosarcoma, malignant giant cell tumor, myeloma bone disease, osteosarcoma, breast cancer (hormone dependent, hormone independent), gynecological cancers (cervical, endometrial, fallopian tube, gestational trophoblastic disease, ovarian, peritoneal, uterine, vaginal and vulvar), basal cell carcinoma (BCC), squamous cell carcinoma (SCC), malignant melanoma, dermatofibrosarcoma protuberans, Merkel cell carcinoma, Kaposi's sarcoma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, malignant mesothelioma (peritoneal mesothelioma, pericardial mesothelioma, pleural mesothelioma), gastro-entero-pancreatic or gastroenteropancreatic neuroendocrine tumor (GEP-NET), carcinoid, pancreatic endocrine tumor (PET), colorectal adenocarcinoma, colorectal carcinoma, aggressive neuroendocrine tumor, leiomyosarcoma, mucinous adenocarcinoma, Signet Ring cell adenocarcinoma, hepatocellular carcinoma, cholangiocarcinoma, hepatoblastoma, hemangioma, hepatic adenoma, focal nodular hyperplasia (nodular regenerative hyperplasia, hamartoma), non-small cell lung carcinoma (NSCLC) (squamous cell lung carcinoma, adenocarcinoma, large cell lung carcinoma), small cell lung carcinoma, thyroid carcinoma, prostate cancer (hormone refractory, androgen independent, androgen dependent, hormone-insensitive), renal cell carcinoma, and soft tissue sarcomas (fibrosarcoma, malignant fibrous hystiocytoma, dermatofibrosarcoma, liposarcoma, rhabdomyosarcoma leiomyosarcoma, hemangiosarcoma, synovial sarcoma, malignant peripheral nerve sheath tumor/neurofibrosarcoma, extraskeletal osteosarcoma).

Many tumors of the haematopoietic and lymphoid tissues are characterized by an increase in cell proliferation, or a particular type of cell. The chronic myeloproliferative diseases (CMPDs) are clonal haematopoietic stem cell disorders characterized by proliferation in the bone marrow of one or more of the myeloid lineages, resulting in increased numbers of granulocytes, red blood cells and/or platelets in the peripheral blood. As such, the use of proteasome inhibitors for the treatment of such diseases is attractive and being examined (Cilloni et al., Haematologica (2007) 92: 1124-1229). CMPD can include chronic myelogenous leukaemia, chronic neutrophilic leukaemia, chronic eosinophilic leukaemia, polycythaemia vera, chronic idiopathic myelofibrosis, essential thrombocythaemia and unclassifiable chronic myeloproliferative disease. An aspect of the invention is the method of treating CMPD comprising administering to a subject in need of such treatment an effective amount of a proteasome inhibitor compound disclosed herein.

Myelodysplastic/myeloproliferative diseases, such as chronic myelomonocytic leukaemia, atypical chronic myeloid leukemia, juvenile myelomonocytic leukaemia and unclassifiable myelodysplastic/myeloproliferative disease, are characterized by hypercellularity of the bone marrow due to proliferation in one or more of the myeloid lineages. Inhibiting the proteasome with a compound or composition as described herein can serve to treat these myelodysplatic/myeloproliferative diseases by providing a subject in need of such treatment an effective amount of the compound or composition.

Myelodysplastic syndromes (MDS) refer to a group of hematopoietic stem cell disorders characterized by dysplasia and ineffective haematopoiesis in one or more of the major myeloid cell lines. Targeting NF-□B with a proteasome inhibitor in these hematologic malignancies induces apoptosis, thereby killing the malignant cell (Braun et al. Cell Death and Differentiation (2006) 13:748-758). A further embodiment of the invention is a method to treat MDS comprising administering to a subject in need of such treatment an effective amount of a compound disclosed herein. MDS includes refractory anemia, refractory anemia with ringed sideroblasts, refractory cytopenia with multilineage dysplasia, refractory anemia with excess blasts, unclassifiable myelodysplastic syndrome and myelodysplastic syndrome associated with isolated del(5q) chromosome abnormality.

Mastocytosis is a proliferation of mast cells and their subsequent accumulation in one or more organ systems. Mastocytosis includes, but is not limited to, cutaneous mastocytosis, indolent systemic mastocytosis (ISM), systemic mastocytosis with associated clonal haematological non-mast-cell-lineage disease (SM-AHNMD), aggressive systemic mastocytosis (ASM), mast cell leukemia (MCL), mast cell sarcoma (MCS) and extracutaneous mastocytoma. Another embodiment of the invention is a method to treat mastocytosis, comprising administering an effective amount of a compound or composition disclosed herein to a subject diagnosed with mastocytosis.

The proteasome regulates NF-κB, which in turn regulates genes involved in the immune and inflammatory response. For example, NF-κB is required for the expression of the immunoglobulin light chain κ gene, the IL-2 receptor α-chain gene, the class I major histocompatibility complex gene, and a number of cytokine genes encoding, for example, IL-2, IL-6, granulocyte colony-stimulating factor, and IFN-β (Palombella et al., Cell (1994) 78:773-785). Thus, in certain embodiments, the invention relates to methods of affecting the level of expression of IL-2, MHC-I, IL-6, TNFα, IFN-β or any of the other previously-mentioned proteins, each method comprising administering to a subject an effective amount of a proteasome inhibitor compound or composition disclosed herein. In certain embodiments, the invention includes a method of treating an autoimmune disease in a mammal comprising administering a therapeutically effective amount of a compound or composition described herein. An “autoimmune disease” herein is a disease or disorder arising from and directed against an individual's own tissues. Examples of autoimmune diseases or disorders include, but are not limited to, inflammatory responses such as inflammatory skin diseases including psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g., Type I diabetes mellitus or insulin dependent diabetes mellitus); multiple sclerosis; Reynaud's syndrome; autoimmune thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Beheet disease; giant cell arteritis; immune complex nephritis; IgA nephropathy; IgM polyneuropathies; immune thrombocytopenic purpura (ITP) or autoimmune thrombocytopenia.

The immune system screens for autologous cells that are virally infected, have undergone oncogenic transformation, or present unfamiliar peptides on their surface. Intracellular proteolysis generates small peptides for presentation to T-lymphocytes to induce MHC class I-mediated immune responses. Thus, in certain embodiments, the invention relates to a method of using the compound as an immunomodulatory agent for inhibiting or altering antigen presentation in a cell, comprising exposing the cell (or administering to a subject) to a compound described herein. Specific embodiments include a method of treating graft or transplant-related diseases, such as graft-versus-host disease or host versus-graft disease in a mammal, comprising administering a therapeutically effective amount of a compound described herein. The term “graft” as used herein refers to biological material derived from a donor for transplantation into a recipient. Grafts include such diverse material as, for example, isolated cells such as islet cells; tissue such as the amniotic membrane of a newborn, bone marrow, hematopoietic precursor cells, and ocular tissue, such as corneal tissue; and organs such as skin, heart, liver, spleen, pancreas, thyroid lobe, lung, kidney, tubular organs (e.g., intestine, blood vessels, or esophagus). The tubular organs can be used to replace damaged portions of esophagus, blood vessels, or bile duct. The skin grafts can be used not only for burns, but also as a dressing to damaged intestine or to close certain defects such as diaphragmatic hernia. The graft is derived from any mammalian source, including human, whether from cadavers or living donors. In some cases, the donor and recipient is the same mammal. Preferably the graft is bone marrow or an organ such as heart and the donor of the graft and the host are matched for HLA class II antigens.

Histiocytic and dendritic cell neoplasms are derived from phagocytes and accessory cells, which have major roles in the processing and presentation of antigens to lymphocytes. Depleting the proteasome content in dendritic cells has been shown to alter their antigen-induced responses (Chapatte et al. Cancer Res. (2006) 66:5461-5468). Thus, another embodiment of the invention comprises administering an effective amount of a compound or composition disclosed herein to a subject with histiocytic or dendritic cell neoplasm. Histiocytic and dendritic cell neoplasms include histiocytic sarcoma, Langerhans cell histiocytosis, Langerhans cell sarcoma, interdigitating dendritic cell sarcoma/tumor, follicular dendritic cell sarcoma/tumor and non-specified dendritic cell sarcoma.

Inhibition of the proteasome has been shown to be beneficial to treat diseases whereby a cell type is proliferating and immune disorders; thus, an embodiment of the invention includes the treatment of lymphoproliferative diseases (LPD) associated with primary immune disorders (PID) comprising administering an effective amount of the disclosed compound to a subject in need thereof. The most common clinical settings of immunodeficiency associated with an increased incidence of lymphoproliferative disorders, including B-cell and T-cell neoplasms and lymphomas, are primary immunodeficiency syndromes and other primary immune disorders, infection with the human immunodeficiency virus (HIV), iatrogenic immunosuppression in patients who have received solid organ or bone marrow allografts, and iatrogenic immunosuppression associated with methotrexate treatment. Other PIDs commonly associated with LPDs, but not limited to, are ataxia telangiectasia (AT), Wiskott-Aldrich syndrome (WAS), common variable immunodeficiency (CVID), severe combined immunodeficiency (SCID), X-linked lymphoproliferative disorder (XLP), Nijmegen breakage syndrome (NBS), hyper-IgM syndrome, and autoimmune lymphoproliferative syndrome (ALPS).

Additional embodiments of the invention relate to methods for affecting the proteasome-dependent regulation of oncoproteins and methods of treating or inhibiting cancer growth, each method comprising exposing a cell (in vivo, e.g., in a subject, or in vitro) to the proteasome inhibitor composition disclosed herein. HPV-16 and HPV-18-derived E6 proteins stimulate ATP- and ubiquitin-dependent conjugation and degradation of p53 in crude reticulocyte lysates. The recessive oncogene p53 has been shown to accumulate at the nonpermissive temperature in a cell line with a mutated thermolabile E1. Elevated levels of p53 may lead to apoptosis. Examples of proto-oncoproteins degraded by the ubiquitin system include c-Mos, c-Fos, and c-Jun. In certain embodiments, the invention relates to a method for treating p53-related apoptosis, comprising administering to a subject an effective amount of a proteasome inhibitor composition disclosed herein.

Another aspect of the invention relates to the use of proteasome inhibitor compositions disclosed herein for the treatment of neurodegenerative diseases and conditions, including, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis).

Alzheimer's disease is characterized by extracellular deposits of β-amyloid protein (β-AP) in senile plaques and cerebral vessels. β-AP is a peptide fragment of 39 to 42 amino acids derived from an amyloid protein precursor (APP). At least three isoforms of APP are known (695, 751, and 770 amino acids). Alternative splicing of mRNA generates the isoforms; normal processing affects a portion of the β-AP sequence, thereby preventing the generation of β-AP. It is believed that abnormal protein processing by the proteasome contributes to the abundance of β-AP in the Alzheimer brain. The APP-processing enzyme in rats contains about ten different subunits (22 kDa-32 kDa). The 25 kDa subunit has an N-terminal sequence of X-Gln-Asn-Pro-Met-X-Thr-Gly-Thr-Ser, which is identical to the β-subunit of human macropain (Kojima, S. et al., Fed. Eur. Biochem. Soc., (1992) 304:57-60). The APP-processing enzyme cleaves at the Gln¹⁵--Lys¹⁶ bond; in the presence of calcium ion, the enzyme also cleaves at the Met⁻¹--Asp¹ bond and the Asp¹--Ala² bond to release the extracellular domain of β-AP.

One aspect of the invention, therefore, relates to a method of treating Alzheimer's disease, comprising administering to a subject an effective amount of a proteasome inhibitor compound or composition disclosed herein. Such treatment includes reducing the rate of β-AP processing, reducing the rate of β-AP plaque formation, reducing the rate of β-AP generation, and reducing the clinical signs of Alzheimer's disease.

In some embodiments, a proteasome inhibitor compound or composition disclosed herein can be useful for treating amyloidosis. Accordingly, provided herein is a method for treating amyloidosis is a subject, comprising administering to a subject an effective amount of a proteasome inhibitor compound or composition disclosed herein.

Fibrosis is the excessive and persistent formation of fibrous connective tissue resulting from the hyperproliferative growth of fibroblasts and is associated with activation of the TGF-β signaling pathway. Fibrosis involves extensive deposition of extracellular matrix and can occur within virtually any tissue or across several different tissues. Normally, the level of intracellular signaling protein (Smad) that activates transcription of target genes upon TGF-β stimulation is regulated by proteasome activity (Xu et al., 2000). However, accelerated degradation of the TGF-β signaling components has been observed in fibrotic conditions, such as cystic fibrosis, injection fibrosis, endomyocardial fibrosis, idiopathic pulmonary fibrosis, myelofibrosis, retroperitoneal fibrosis, progressive massive fibrosis, nephrogenic systemic fibrosis. Other conditions that are often associated with fibrosis include cirrhosis, diffuse parenchymal lung disease, post-vasectomy pain syndrome, tuberculosis, sickle-cell anemia and rheumatoid arthritis. An embodiment of the invention is the method of treating a fibrotic or fibrotic-associated condition comprising administering an effective amount of the composition described herein to a subject in need of such treatment.

The treatment of burn victims is often hampered by fibrosis. Thus, in certain embodiments, the invention relates to the topical or systemic administration of a subject inhibitor to treat burns. Wound closure following surgery is often associated with disfiguring scars, which may be prevented by inhibition of fibrosis. Thus, in certain embodiments, the invention relates to a method for the prevention or reduction of scarring.

Overproduction of lipopolysaccharide (LPS)-induced cytokines such as TNFα is considered to be central to the processes associated with septic shock. Furthermore, it is generally accepted that the first step in the activation of cells by LPS is the binding of LPS to specific membrane receptors. The α- and β-subunits of the 20S proteasome complex have been identified as LPS-binding proteins, suggesting that the LPS-induced signal transduction may be an important therapeutic target in the treatment or prevention of sepsis (Qureshi, N. et al., J. Immun. (2003) 171: 1515-1525). Therefore, in certain embodiments, the proteasome inhibitor composition may be used for the inhibition of TNFα to prevent and/or treat septic shock.

Ischemia and reperfusion injury results in hypoxia, a condition in which there is a deficiency of oxygen reaching the tissues of the body. This condition causes increased degradation of Iκ-Bα, thereby resulting in the activation of NF-κB (Koong et al., 1994). It has been demonstrated that the severity of injury resulting in hypoxia can be reduced with the administration of a proteasome inhibitor (Gao, et al., 2000; Bao, et al., 2001; Pye, et al., 2003). Therefore, certain embodiments of the invention relate to a method of treating an ischemic condition or reperfusion injury comprising administering to a subject in need of such treatment an effective amount of the proteasome inhibitor compound disclosed herein. Examples of such conditions or injuries include, but are not limited to, acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

NF-κB also binds specifically to the HIV-enhancer/promoter. When compared to the Nef of mac239, the HIV regulatory protein Nef of pbj14 differs by two amino acids in the region which controls protein kinase binding. It is believed that the protein kinase signals the phosphorylation of IκB, triggering IκB degradation through the ubiquitin-proteasome pathway. After degradation, NF-κB is released into the nucleus, thus enhancing the transcription of HIV (Cohen, J., Science, (1995) 267:960). In certain embodiments, the invention relates to a method for inhibiting or reducing HIV infection in a subject, or a method for decreasing the level of viral gene expression, each method comprising administering to the subject an effective amount of a proteasome inhibitor compound or composition disclosed herein.

Viral infections contribute to the pathology of many diseases. Heart conditions such as ongoing myocarditis and dilated cardiomyopathy have been linked to the coxsackievirus B3. In a comparative whole-genome microarray analyses of infected mouse hearts, specific proteasome subunits were uniformly up-regulated in hearts of mice which developed chronic myocarditis (Szalay et al, Am J Pathol 168:1542-52, 2006). Some viruses utilize the ubiquitin-proteasome system in the viral entry step where the virus is released from the endosome into the cytosol. The mouse hepatitis virus (MHV) belongs to the Coronaviridae family, which also includes the severe acute respiratory syndrome (SARS) coronavirus. Yu and Lai (J Virol 79:644-648, 2005) demonstrated that treatment of cells infected with MHV with a proteasome inhibitor resulted in a decrease in viral replication, correlating with reduced viral titer as compared to that of untreated cells. The human hepatitis B virus (HBV), a member of the Hepadnaviridae virus family, likewise requires virally encoded envelop proteins to propagate. Inhibiting the proteasome degradation pathway causes a significant reduction in the amount of secreted envelope proteins (Simsek et al, J Virol 79:12914-12920, 2005). In addition to HBV, other hepatitis viruses (A, C, D and E) may also utilize the ubiquitin-proteasome degradation pathway for secretion, morphogenesis and pathogenesis. Accordingly, in certain embodiments, the invention relates to a method for treating viral infection, such as SARS or hepatitis A, B, C, D and E, comprising contacting a cell with (or administering to a subject) an effective amount of a compound or composition disclosed herein.

In certain embodiments, the disclosed compositions may be useful for the treatment of a parasitic infection, such as infections caused by protozoan parasites. The proteasome of these parasites is considered to be involved primarily in cell differentiation and replication activities (Paugam et al., Trends Parasitol. 2003, 19(2): 55-59). Furthermore, entamoeba species have been shown to lose encystation capacity when exposed to proteasome inhibitors (Gonzales, et al., Arch. Med. Res. 1997, 28, Spec No: 139-140). In certain such embodiments, the administrative protocols for the proteasome inhibitor compositions are useful for the treatment of parasitic infections in humans caused by a protozoan parasite selected from Plasmodium sps. (including P. falciparum, P. vivax, P. malariae, and P. ovale, which cause malaria), Trypanosoma sps. (including T. cruzi, which causes Chagas' disease, and T. brucei which causes African sleeping sickness), Leishmania sps. (including L. amazonesis, L. donovani, L. infantum, L. mexicana, etc.), Pneumocystis carinii (a protozoan known to cause pneumonia in AIDS and other immunosuppressed patients), Toxoplasma gondii, Entamoeba histolytica, Entamoeba invadens, and Giardia lamblia.

In certain embodiments, the disclosed proteasome inhibitor compositions are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds that act as proteasome inhibitors in the treatment of parasitic diseases are described in WO 98/10779, which is incorporated herein in its entirety.

In certain embodiments, the proteasome inhibitor compositions inhibit proteasome activity in a parasite without recovery in red blood cells and white blood cells. In certain such embodiments, the long half-life of blood cells may provide prolonged protection with regard to therapy against recurring exposures to parasites. In certain embodiments, the proteasome inhibitor compositions may provide prolonged protection with regard to chemoprophylaxis against future infection.

Prokaryotes have an equivalent to the eukaryote 20S proteasome particle. Although the subunit composition of the prokaryote 20S particle is simpler than that of eukaryotes, it has the ability to hydrolyze peptide bonds in a similar manner. For example, the nucleophilic attack on the peptide bond occurs through the threonine residue on the N-terminus of the β-subunits. Thus, an embodiment of this invention relates to a method of treating prokaryotic infections, comprising administering to a subject an effective amount of a proteasome inhibitor compound or composition disclosed herein. Prokaryotic infections may include diseases caused by either mycobacteria (such as tuberculosis, leprosy or Buruli ulcer) or archaebacteria.

It has also been demonstrated that inhibitors that bind to the 20S proteasome stimulate bone formation in bone organ cultures. Furthermore, when such inhibitors have been administered systemically to mice, certain proteasome inhibitors increased bone volume and bone formation rates over 70% (Garrett, I. R. et al., J. Clin. Invest. (2003) 111: 1771-1782), therefore suggesting that the ubiquitin-proteasome machinery regulates osteoblast differentiation and bone formation. Therefore, a disclosed proteasome inhibitor compound or composition may be useful in the treatment and/or prevention of diseases associated with bone loss, such as osteoporosis.

Thus, in certain embodiments, the invention relates to a method for treating a disease or condition selected from cancer, autoimmune disease, graft or transplant-related condition, neurodegenerative disease, fibrotic-associated condition, ischemic-related conditions, infection (viral, parasitic or prokaryotic) and diseases associated with bone loss, comprising administering a compound or composition as disclosed herein.

Also provided herein is a conjoint therapy wherein one or more other therapeutic agents are administered with a peptide proteasome inhibitor or a pharmaceutical composition comprising a peptide proteasome inhibitor. Such conjoint treatment may be achieved by way of the simultaneous, sequential, or separate dosing of the individual components of the treatment.

In certain embodiments, a composition provided herein (e.g., pharmaceutical compositions that include oprozomib) is conjointly administered with one or more other proteasome inhibitor(s).

In certain embodiments, a composition provided herein (e.g., pharmaceutical compositions that include oprozomib) is conjointly administered with one or more chemotherapeutics. Suitable chemotherapeutics may include, natural products such as vinca alkaloids (i.e. vinblastine, vincristine, and vinorelbine), taxanes (e.g., docetaxel, paclitaxel), epidipodophyllotoxins (i.e. etoposide, teniposide), antibiotics (dactinomycin (actinomycin D) daunorubicin, doxorubicin and idarubicin; e.g., doxorubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, ifosphamide, cyclophosphamide and analogs, melphalan, chlorambucil, e.g., melphalan), ethylenimines and methylmelamines (hexaamethylmelaamine and thiotepa), alkyl sulfonates (busulfan), nitrosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine); aromatase inhibitors (anastrozole, exemestane, and letrozole); platinum coordination complexes (cisplatin, carboplatin), procarbazine, hydroxyurea, mitotane, aminoglutethimide; DNA binding/Cytotoxic agents (e.g., Zalypsis); histone deacetylase (HDAC) inhibitors (e.g., trichostatin, sodium butyrate, apicidan, suberoyl anilide hydroamic acid (SAHA (Vorinostat)), trichostatin A, depsipeptide, apicidin, A-161906, scriptaid, PXD-101, CHAP, butyric acid, depudecin, oxamflatin, phenylbutyrate, valproic acid, MS275 (N-(2-Aminophenyl)-44N-(pyridine-3-ylmethoxy-carbonyl)aminomethyllbenzamide), LAQ824/LBH589, CI994, MGCD0103, ACY-1215, Panobinostat); hormones (i.e. estrogen) and hormone agonists such as leutinizing hormone releasing hormone (LHRH) agonists (goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, a pharmaceutical composition as provided herein (e.g., pharmaceutical compositions that include oprozomib) is conjointly administered with one or more histone deacetylase (HDAC) inhibitors (e.g., trichostatin, sodium butyrate, apicidan, suberoyl anilide hydroamic acid (“SAHA” (Vorinostat)), trichostatin A, depsipeptide, apicidin, A-161906, scriptaid, PXD-101, CHAP, butyric acid, depudecin, oxamflatin, phenylbutyrate, valproic acid, MS275 (N-(2-Aminophenyl)-44N-(pyridine-3-ylmethoxy-carbonyl)aminomethyllbenzamide), LAQ824/LBH589, CI994, MGCD0103, ACY-1215, Panobinostat; e.g., SAHA, ACY-1215, Panobinostat).

In certain embodiments, a pharmaceutical composition as provided herein (e.g., pharmaceutical compositions that include oprozomib) is conjointly administered with one or more nitrogen mustards (mechlorethamine, ifosphamide, cyclophosphamide and analogs, melphalan, chlorambucil, e.g., melphalan).

In certain embodiments, a pharmaceutical composition as provided herein (e.g., pharmaceutical compositions that include oprozomib) is conjointly administered with one or more DNA binding/Cytotoxic agents (e.g., Zalypsis).

In certain embodiments, a pharmaceutical composition as provided herein (e.g., pharmaceutical compositions that include oprozomib) is conjointly administered with one or more taxanes (e.g., docetaxel, paclitaxel).

In certain embodiments, a pharmaceutical composition as provided (e.g., pharmaceutical compositions that include oprozomib) is conjointly administered with one or more antibiotics (dactinomycin (actinomycin D) daunorubicin, doxorubicin and idarubicin; e.g., doxorubicin).

In some embodiments, a pharmaceutical composition as provided herein (e.g., pharmaceutical compositions that include oprozomib) is conjointly administered with one or more cytokines. Cytokines include, but are not limited to, Interferon-γ, -α, and -β, Interleukins 1-8, 10 and 12, Granulocyte Monocyte Colony-Stimulating factor (GM-CSF), TNF-α and -β, and TGF-β.

In some embodiments, a pharmaceutical composition provided herein (e.g., pharmaceutical compositions that include oprozomib) is conjointly administered with one or more steroids. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof (e.g., hydrocortisone, dexamethasone, methylprednisolone and prednisolone; e.g., dexamethasone).

The invention will be further described in the following examples. It should be understood that these examples are for illustrative purposes only and are not to be construed as limiting this invention in any manner.

### EXAMPLES

### Example 1. Preparation of Oprozomib (Compound 1 in the Example Below)

### Synthesis of Compound 1

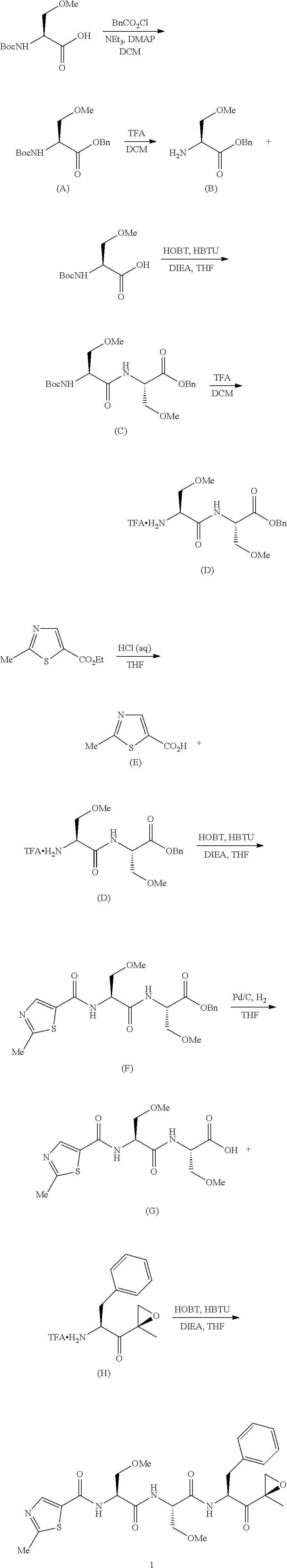

### Synthesis of (A)

To a 0° C. solution of N-Boc serine(methyl ether) (43.8 g, 200 mmol), triethylamine (26.5 g, 260 mmol) and 4-(dimethylamino)pyridine in dichloromethane (1.2 L) was added a solution of benzyl chloroformate (41 g, 240 mmol) in dichloromethane (250 mL) over 30 minutes. The resulting mixture was stirred at the same temperature for another 3 hours. Saturated aqueous sodium bicarbonate (200 mL) was added and organic layer was separated, the residual mixture was extracted with dichloromethane (2×400 mL). The combined organic layers were washed with saturated aqueous sodium bicarbonate (200 mL) and brine (200 mL), dried over sodium sulfate and filtered through Celite-545. The solvents were removed under reduced pressure and residue was purified by flash chromatography (silica gel, hexane and ethyl acetate). Compound (A) (54 g) was isolated and characterized by LC/MS (LRMS (MH) m/z: 310.16).

### Synthesis of (B)

To a 0° C. solution of Compound (A) (54 g) in dichloromethane (200 mL) was added trifluoroacetic acid (200 mL) over 10 minutes, and the resulting mixture was stirred at the same temperature for another 3 hours. The solvents were removed under reduced pressure and the residue was placed under high vacuum overnight giving the TFA salt of Compound (B), which was characterized by LC/MS (LRMS (MH) m/z: 210.11).

### Synthesis of (C)

To a 0° C. solution of Compound (B) (43.8 g, 200 mmol), N-Boc serine(methyl ether) (36.7 g, 167 mmol), HOBT (27 g, 200 mmol) and HBTU (71.4 g, 200 mmol) in tetrahydrofuran (1.2 L) was added a solution of N,N-diethylisopropylamine (75 g, 600 mmol) in tetrahydrofuran (250 mL) over 10 minutes, and the pH of the resulting mixture was ˜8. The mixture was stirred at room temperature for another 5 hours. Most of the solvent were removed under reduced pressure at room temperature and diluted with saturated aqueous sodium bicarbonate (400 mL). Then it was extracted with ethyl acetate (3×400 mL), washed with sodium bicarbonate (100 mL) and brine (100 mL). The combined organic layers were dried over sodium sulfate and filtered through Celite-545°. The solvents were removed under reduced pressure and residue was purified by flash chromatography (silica gel, hexane and ethyl acetate). Compound (C) (65 g) was isolated and characterized by LC/MS (LRMS (MH) m/z: 411.21).

### Synthesis of (D)

To a 0° C. solution of Compound (C) (18 g) in dichloromethane (100 mL) was added trifluoroacetic acid (80 mL) over 5 minutes, and the resulting mixture was stirred at the same temperature for another 3 hours. The solvents were removed under reduced pressure and the residue was placed under high vacuum overnight giving the TFA salt of intermediate (D), which was characterized by LC/MS (LRMS (MH) m/z: 311.15).

### Synthesis of (E)

To a 0° C. solution of ethyl 2-methyl-thiazole-5-carboxylate (15 g, 88 mmol) in tetrahydrofuran (50 mL) was added aqueous sodium hydroxide solution (5 N, 50 mL) over 10 minutes, and the resulting solution was stirred at room temperature for another 2 hours. It was then acidified with hydrochloric acid (2 N) to pH=1 and extracted with tetrahydrofuran (3×100 mL). The combined organic layers were washed with brine (30 mL) and dried over sodium sulfate. Most of the solvents were removed under reduced pressure and the residue was lyophilized to afford Compound (E) (14 g).

### Synthesis of (F)

To a 0° C. solution of Compound (D) (41 mmol) and 2-methyl-thiazole-5-carboxylic acid (E) (6.0 g, 42 mmol), HOBT (7.9 g, 50 mmol) and HBTU (18.0 g, 50 mmol) in tetrahydrofuran (800 mL) was added a solution of N,N-diethylisopropylamine (˜50 g) in tetrahydrofuran (200 mL) over 5 minutes until its pH reached approximately 8.5. The resulting mixture was stirred at same temperature overnight. It was then quenched with saturated aqueous sodium bicarbonate solution (200 mL), and most of the solvents were removed under reduced pressure. The residual mixture was extracted with ethyl acetate (3×400 mL). The combined organic layers were washed with saturated aqueous sodium bicarbonate (200 mL) and brine (100 mL), dried over sodium sulfate and filtered through Celite-545®. The solvents were removed under reduced pressure and residue was purified by flash chromatography (silica gel, ethyl acetate with 2% methanol). Compound (F) (17.1 g) was isolated and characterized by LC/MS (LRMS (MH) m/z: 436.15).

### Synthesis of (G)

To a solution of Compound (F) (17.1 g, 95 mmol) in methanol (300 mL) was added 10% Pd/C (3 g). The resulting mixture was allowed to stir under 1 atmosphere of hydrogen for 48 hours. The mixture was filtered through Celite 545° and the filter cake was washed with methanol (˜200 mL). The organic layers were concentrated under reduced pressure and placed under high vacuum to yield Compound (G), which was characterized by LC/MS (LRMS (MH) m/z: 346.1).

### Synthesis of (H)

N-Boc phenylalanine-ketoepoxide (140 mg, 0.46 mmol) was diluted with DCM (2 mL) and cooled to 0° C. To this solution was added trifluoroacetic acid (6 mL). The cooling bath was removed and the reaction stirred for 1 hour at which time TLC showed complete consumption of starting material. The resulting solution was concentrated under reduced pressure and placed under high vacuum to yield TFA salt of Compound (H).

### Synthesis of Compound 1

To a 0° C. solution of aforementioned Compounds (H) (131 mg, 0.38 mmol) and (J) (0.46 mmol), HOBT (75 mg, 0.48 mmol) and HBTU (171 mg, 0.48 mmol) in tetrahydrofuran (20 mL) and N,N-dimethylformamide (10 mL) was added N,N-diethylisopropylamine (1 mL) dropwise. The mixture was stirred at the same temperature for another 5 hours. It was then quenched with saturated aqueous sodium bicarbonate solution (20 mL), and most of the solvents were removed under reduced pressure. The residual mixture was then extracted with ethyl acetate (3×40 mL). The combined organic layers were washed with saturated aqueous sodium bicarbonate (20 mL) and brine (10 mL), dried over sodium sulfate and filtered through Celite-545. The solvents were removed under reduced pressure and residue was purified by HPLC (0.02 M aqueous ammonium acetate and acetonitrile (66/34) to afford Compound 1 (92 mg), which was lyophilized and characterized by LC/MS (LRMS (MET) m/z: 533.2).

### Example 2

Amorphous Compound 1 (50 mg) was dissolved in acetonitrile (1 mL), then deionized water (2 mL) was added, and the solution brought to supersaturation by slowly evaporating off 1 mL over about 1-2 weeks. The resulting crystals were filtered, washed with 1 mL 1:2 acetonitrile-water, and dried under vacuum for 12 hours to provide a crystalline polymorph of Compound 1 (25 mg) with a melting point of 148° C. The characteristic DSC curve of the sample is shown in FIG. 3 as recorded on a TA Instruments Differential Scanning calorimeter 2920® at a heating rate of 10° C./minute.

### Example 3

Amorphous Compound 1 (611 mg) was dissolved in tetrahydrofuran (5 mL), followed by addition of hexanes (5 mL) and the solution was seeded with crystalline polymorph Compound 1 as prepared in Example 2, and the solution brought to supersaturation by slowly evaporating off 5 mL over about 17 hours. The resulting crystals were filtered, washed with 1 mL 1:1 tetrahydrofuran-hexanes, and dried under vacuum for 12 hours to provide a crystalline polymorph of Compound 1 (150 mg) with a melting point of 147° C.

### Example 4

Amorphous Compound 1 (176 mg) was dissolved in tetrahydrofuran (5 mL), then toluene (25 mL) was added. The solution was seeded with crystalline polymorph Compound 1 as prepared in Example 2, and the solution was brought to supersaturation by slowly evaporating off 20 mL over about 2 days. The resulting crystals were filtered, washed with 15 mL toluene, and dried under vacuum for 12 hours to provide a crystalline polymorph of Compound 1 (88 mg) with a melting point of 149° C.

### Example 5

Amorphous Compound 1 (312 mg) was dissolved in toluene (50 mL), heated to about 100° C. to complete dissolution, then hexanes (50 mL) were added and the solution was seeded with crystalline polymorph Compound 1 as prepared in Example 2, and the solution brought to supersaturation by slowly evaporating off 60 mL over about 2 days. The resulting crystals were filtered, washed with 10 mL toluene, and dried under vacuum for 12 hours to provide a crystalline polymorph of Compound 1 (156 mg) with a melting point of 149° C.

### Example 6

Amorphous Compound 1 (1.4 g) was dissolved in toluene (25 mL), heated to about 50° C. to complete dissolution, then brought to supersaturation by cooling to 22° C. and allowing the compound to crystallize for 12 hours. The resulting crystals were filtered, washed with 5 mL hexanes, and dried under vacuum for 12 hours to provide a crystalline polymorph of Compound 1 (0.94 g) with a melting point of 149° C.

### Example 7

### Synthesis of Compound 1

### Synthesis of (H)

N-Boc phenylalanine-ketoepoxide (1.0 equivalent) was dissolved in DCM (3 L/kg of N-Boc phenylalanine-ketoepoxide) in a 3-neck round bottom flask under inert atmosphere and the solution was cooled in ice bath. Then, TFA (5.0 equivalents) was added at a rate to maintain the internal temperature below 10° C. The reaction mixture was then warmed to approximately 20° C. and stirred for 1 to 3 hours. MTBE (3.6 L/kg of N-Boc phenylalanine-ketoepoxide) was then added to the reaction mixture while maintaining mixture temperature below 25° C. Heptane (26.4 L/kg of N-Boc phenylalanine-ketoepoxide) was then added the reaction was cooled to between −5 and 0° C. for 2 to 3 hours to allow crystallization of Compound (H). The white solid was filtered and rinsed with heptane (3 L/kg of N-Boc phenylalanine-ketoepoxide). The white solid was then under vacuum for 12 hours at 22° C. Yield obtained was 86%, with HPLC purity 99.4%.

### Synthesis of Compound 1

Compound (H) (1.2 equivalents), Compound (G) (1.0 equivalent), HBTU (1.2 equivalents), HOBT (1.2 equivalents) and N-methyl pyrrolidinone (8 L/kg of Compound (G)) were added to a dry flask under inert atmosphere, and the mixture was stirred at 23° C. to complete dissolution. The reaction was then cooled to between −5 and 0° C., and diisopropylethylamine (2.1 equivalents) was added over 15 minutes, while maintaining an internal reaction temperature of less than 0° C. The reaction mixture was stirred at 0° C. for 12 hours.

Crude Compound 1 was precipitated by pouring the reaction mixture onto 8% sodium bicarbonate (40 L/kg of Compound (G)) and the suspension of crude Compound 1 was stirred for 12 hours at 20 to 25° C., followed by stirring at 0 to 5° C. for 1 hour. The white solid was filtered and rinsed with water (5 L/kg of Compound (G)). The white solid was then reslurried in water (15 L/kg) for 3 hours at 20 to 25° C., filtered and rinsed with water (5 L/kg of Compound (G)) and isopropyl acetate (2×2 L/kg of Compound (G)). The white solid was dried under vacuum at 45° C. to constant weight. Yield of crude Compound 1 was 65%, with HPLC purity of 97.2%.

Crude Compound 1 was completely dissolved in isopropyl acetate (20 L/kg of crude Compound 1) by stirring and heating at 85° C. The solution was then hot filtered to remove any particulate matter and the solution was re-heated to 85° C. to provide clear solution. The clear solution was allowed to cool at 10° C. per hour to 65° C. before adding seed crystals. The solution was allowed to cool at 10° C. per hour to 20° C., when substantial crystallization of Compound 1 occurred. The suspension was stirred at 20° C. for 6 hours, followed by stirring at 0 to 5° C. for a minimum of 2 hours and filtration and rinsing with isopropyl acetate (1 L/kg of crude Compound 1). The purified Compound 1 was dried under vacuum at 45° C. for a minimum of 24 hours to constant weight. Yield of Compound 1 was 87%, with HPLC purity 97.2%.

### Example 8

### Synthesis of Compound 1

Compound (H) (1.1 equivalents), Compound (G) (1.0 equivalent), HBTU (1.5 equivalents), HOBT (1.5 equivalents) and DMF (8 L/kg of Compound (G)) were added to a dry flask under inert atmosphere, and the mixture was stirred at 23° C. to complete dissolution. The reaction was then cooled to between −5 and 0° C., and diisopropylethylamine (2.1 equivalents) was added over 15 minutes, while maintaining an internal reaction temperature of less than 0° C. The reaction mixture was then stirred at 0° C. for 3 hours.

The reaction mixture was quenched by addition of pre-chilled saturated sodium bicarbonate (94 L/kg of Compound (G)), while maintaining internal temperature of less 10° C. The content was then transferred to a separatory funnel. The mixture was extracted with ethyl acetate (24 L/kg of Compound (G)), and the organic layer was washed with saturated sodium bicarbonate (12 L/kg of Compound (G)) and with saturated sodium chloride (12 L/kg of Compound (G)).

The organic layer was concentrated under reduced pressure with a bath temperature of less than 30° C. to 15 L/kg of Compound (G), followed by co-distillation with isopropyl acetate (2×24 L/kg of PR-022). Final volume was adjusted to 82 L/kg of Compound (G) with isopropyl acetate before heating to 60° C. to obtain a clear solution. The clear solution mixture was allowed to cool to 50° C. before adding seed crystals. The solution was allowed to cool to 20° C., when substantial crystallization of Compound 1 had occurred. The suspension was stirred at 0° C. for 12 hours before filtration and rinsing with isopropyl acetate (2 L/kg of Compound 1). Compound 1 was dried under vacuum at 20° C. for 12 hours to constant weight. Yield of Compound 1 was 48%, with HPLC purity of 97.4%.

### Example 2. Preparation and Analysis of Oprozomib Tablets

Following is a general procedure followed to prepare tablet, granulation and compress tablets. Oprozomib 25 mg, 50 mg and 200 mg Immediate Release (IR) tablets are manufactured via dry granulation using a roller compaction process

Dry Granulation
Screen Oprozomib and microcrystalline celluose in a metal sieve
Blend the screened components with lactose monohydrate and croscarmellose sodium in a tumble blender
Blend pre-screened magnesium stearate with materials from Step 2 in a tumble blender
Compact the blend into ribbons and mill with a roller compactor
Blend granules with pre-screened magnesium stearate in a tumble blender

### Tablet Compression and Coating

Tablets weighing about 78 to about 80 mg, about 150 mg to about 156 mg, and about 600 mg with 25 mg, 50 mg and 200 mg drug loading were compressed with round standard concave 9/32″, 13/32″ or 15/32″ tooling respectively using a Carver Press or single station press or a rotary tablet press
Tablets were compressed at predetermined pressure and evaluated for thickness and hardness
Tablet characteristics and process parameters are documented
Tablets prepared are stored at room temperature (“RT”) until further processed or used
Tablets were film coated using a perforated pan coater with Opadry II® 85F18422 which is an immediate release coating polymer formulation marketed by Colorcon®

### Tablet Characterization

Tablets prepared were characterized for thickness, hardness, friability and dissolution characteristics. The tablet granulation was characterized for compressibility index and particle size distribution
Thickness was measured using a VWR Electronic Digital Caliper®
Hardness was measured using a Caleva THT-15® hardness tester
Dissolution was performed with USP Type II Paddle apparatus at 75 rpm in pH 5.5 buffer using an Agilent VK 700® dissolution apparatus and VK8000® Dissolution Sampling Station
Dissolution samples were analyzed using an Agilent 1260 Infinity® HPLC system with Agilent 1200® auto sampler and DAD detector

### Example 3. Statistical Analysis

Either student t-tests or ANOVA was performed for the statistical analysis of the data using GraphPad Prism® software when required. Similarity factor (f2) was also calculated to compare the dissolution profiles.

### Example 4. Formulation Release Profiles

[1] Since the tablet formulations were manufactured manually by hand using one of the following: Carver press, single station press, or rotary tablet press, the uniformity of the tablets prepared were monitored by measuring the thickness and weights of all the tablets and hardness on a few of them. The desired tablet thickness was defined to be in the range of about 2.5 millimeters to about 4.0 millimeters as measured by the digital calipers. The tablet can have a thickness of from about 3.0 millimeters to about 3.77 millimeters, e.g., about 3.04 mm; e.g., about 3.75 mm.

Tablets outside the desired thickness range were rejected. The tablet hardness is inversely proportional to the thickness (for the current working range) and the thickness and hardness of the tablets were well correlated. The desired average tablet hardness strength was between about 1.00 to about 25.00 kp, e.g., about 5 kp. e.g., about 8 kp.

### Example 5. Stability Study

The stability of oprozomib tablets prepared and stored at room temperature for more than 1 month were evaluated for assay and impurities and were found to be acceptable without any anomalous peaks implying stability at RT. In some embodiments, the 25 mg and 50 mg formulations of Table 1 are stable upon actual or simulated storage at open conditions at 25° C./60% relative humidity for at least 1 month.

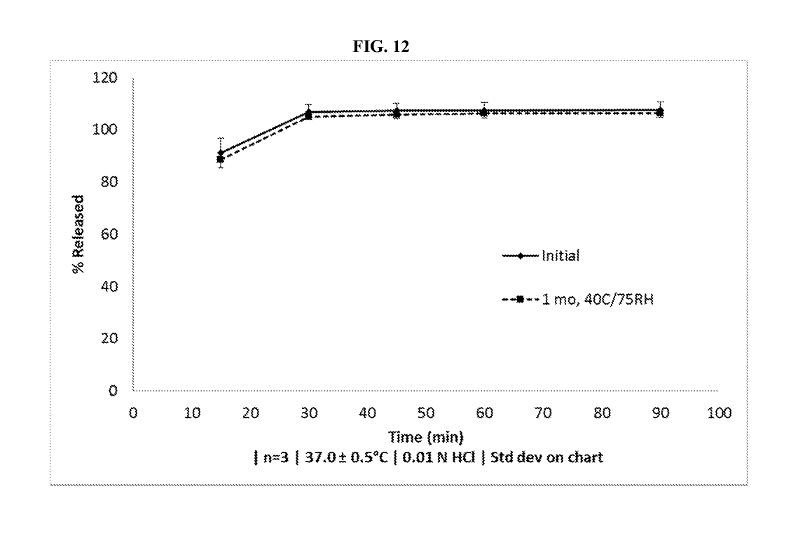

In some embodiments, the 25 mg formulation of Table 1a, which comprises a desiccant, preferably a hydrophilic silica, is stable upon actual or simulated storage at open conditions at 40° C./75% RH for at least 1 month (FIG. 12). After multiple experiments with different grades of silicon dioxide as the desiccant, the most preferred silicon dioxide to employ in the formulation is porous and has a surface area of about 500 to about 1000 m²/g, most preferably about 700 m²/g. The most preferred silicon dioxide has an average pore volume of about 0.2 to about 1 cc/g, most preferably about 0.4 cc/g. The silicon dioxide used in the formulation of Table 1a is Syloid® 63FP, commercially available from W.R. Grace (Columbia, Md.).

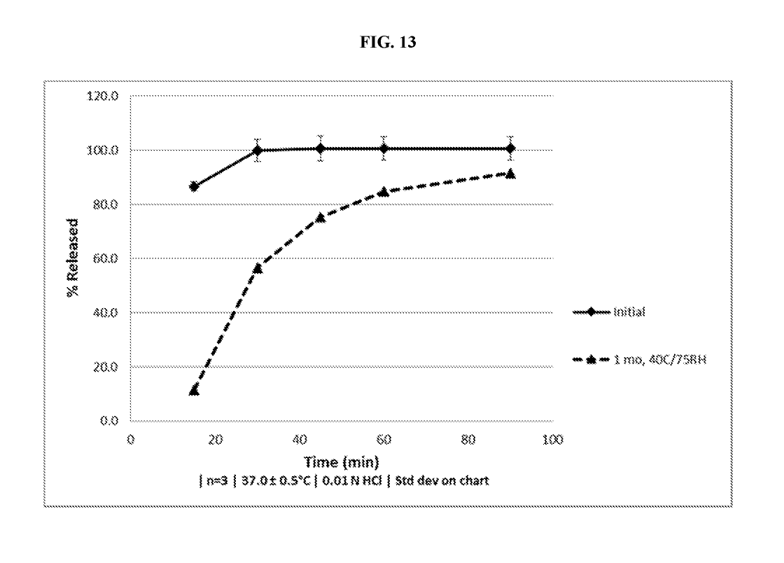

FIG. 13 is a graph showing the different dissolution profiles of experimental formulations of Table 1a using a different grade of silicon dioxide, Cab-o-sil® M-5P (Cabot Corp., Boston, Mass.), than in FIG. 12. When the formulation storage time is T=0 (Initial), the release profile is consistent with previous release profiles of the Table 1a formulation. However, the slow down in release profile of the formulation after T=1 month is inconsistent and not desirable for a commercial formulation due to the NVD adverse events associated with the slow down in the release of oprozomib in the GI tract. Table 7 below compares the properties of the different grades of silicon dioxide employed:

TABLE 7

| Desiccant | | |
|---|---|---|
| (Silicon Dioxide) | Surface Area (m²/g) | Average Pore volume (cc/g) |
| Cab-o-sil | 200 | Nonporous |
| Syloid 63FP | 700 | 0.4 |

Stability studies were carried out using one of the following procedures:
(i) Tablets were packaged in 75 cc white HDPE bottles with closures and stored at 30° C.±2° C./65% relative humidity (RH)±5% RH and 40° C.±2° C./75% RH±5% RH. Tablets were tested for appearance, hardness, assay and impurities and dissolution at pre-determined time points.
(ii) Tablets were packaged in 75 cc white HDPE bottles with closures and desiccant and stored at 5° C.±3° C., 25° C.±2° C./60% relative humidity (RH)±5% RH and 40° C.±2° C./75% RH±5% RH. Tablets were tested for appearance, hardness, assay and impurities and dissolution at pre-determined time points.
(iii) In preferred embodiments, when the formulation is stored in a 75 cc HDPE bottle with desiccant at 5° C.±3° C., for at least 1 month, the formulation shows less than about 1.0% degradation of oprozomib. In more preferred embodiments, the amount of degradation of oprozomib is less than 0.5%, 0.4%, 0.3%, 0.2%, and in some instances, less than 0.1%.
(iv) To investigate and improve the slowdown in the dissolution profile of the formulations of Tables 1 and 1a, with different grades of desiccant (FIGS. 12 and 13), stability studies were conducted under open conditions at 40° C.±2° C./75% RH±5% RH.

### Example 6. PK/PD Studies

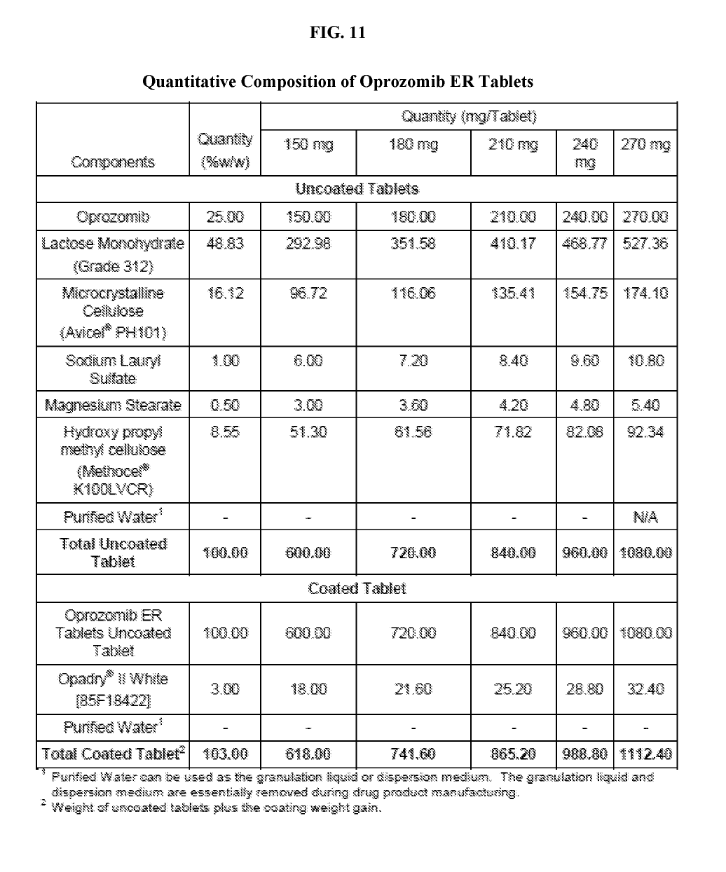

PK/PD studies were conducted using the present IR formulations as described in Table 1: an oral suspension, wherein the suspension is a 1.2 mg/mL oprozomib suspension in 1% carboxymethylcellulose sodium (medium viscosity 400-800 cP) vehicle; and an extended release formulation as described in FIG. 11.

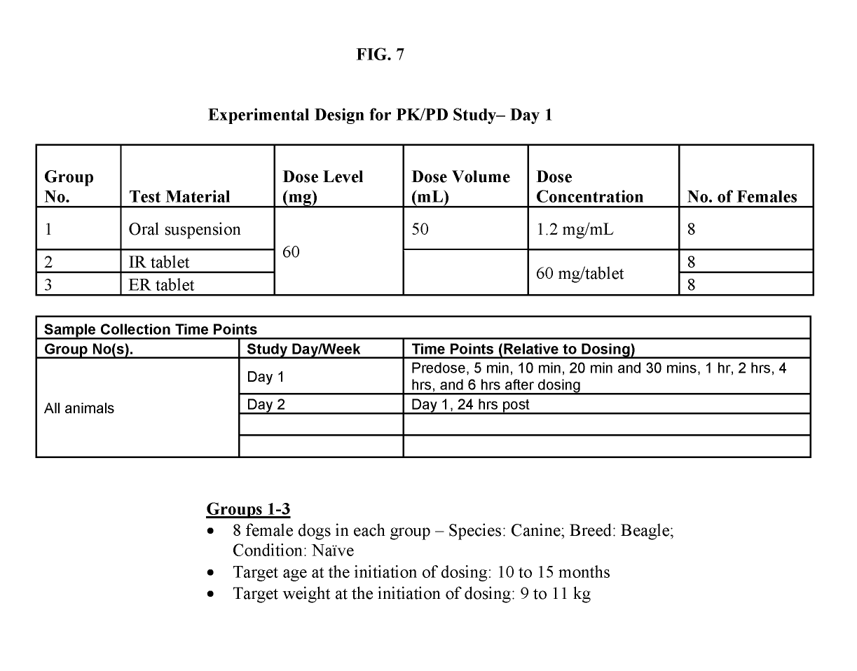

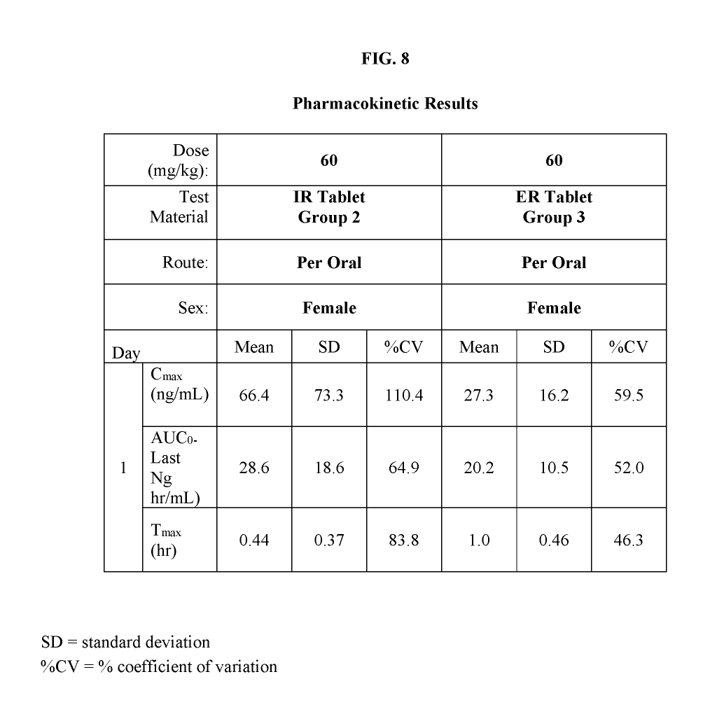

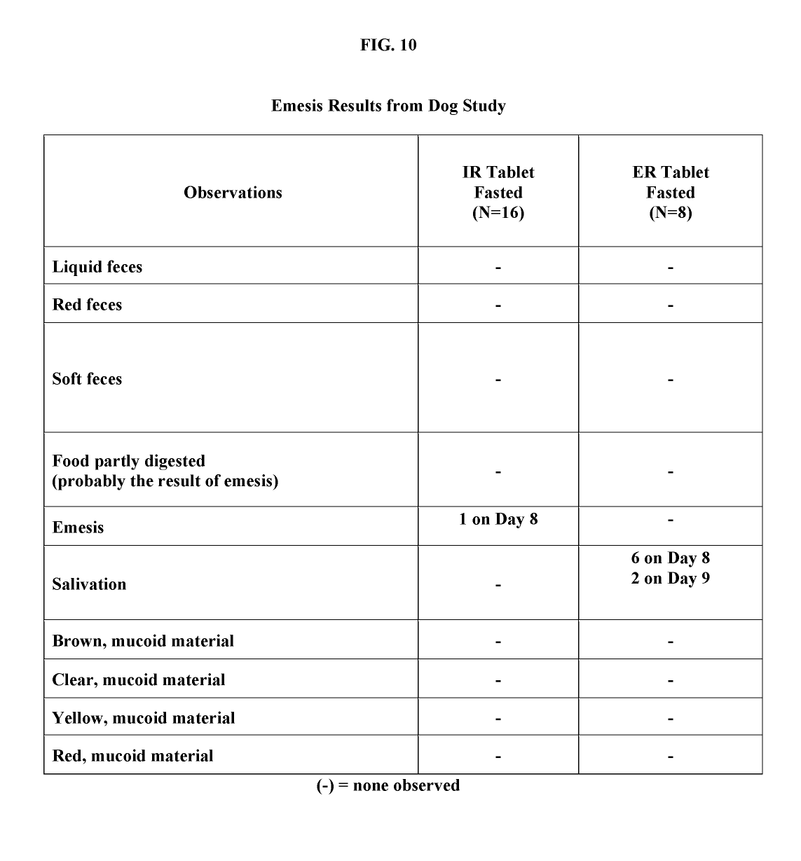

In vivo dog data show that oprozomib administration using IR formulations reduced GI intolerability (such as emesis (vomiting) events and increased salivation) relative to ER formulation while maintaining the PK/PD activity (FIGS. 7, 8, 9 and 10). Female dogs were administered a single dose of 60 mg/kg oprozomib in oral suspension, immediate release, or ER formulations. Blood samples were collected from pre-dose to 24 hours post-dose for plasma PK parameter determination and blood PD analyses of proteasome inhibition (see FIG. 8). GI tolerability were recorded up to 48 hours post-dose (FIG. 10). The area under the plasma concentration curve to the last time point (AUCₗₐₛₜ) and maximum concentration (Cₘₐₓ) exposures were superior for the IR formulation relative to the ER (FIG. 7). The IR formulations had a time to peak plasma concentrations of from about 30 minutes to about 45 minutes. Rapid potent inhibition of proteasome activity (100% of pre-dose) was observed for the IR formulations (FIG. 9). IR formulations caused less GI intolerability than the ER formulations (FIG. 10). Following a 60 mg/kg dose, the immediate formulations caused only one emesis event and no increased salivation event while the ER formulation caused a total of 8 increased salivation events on Days 8 and 9. As such, the formulations described herein can provide a reduced incidence or severity of one or more GI side effects (e.g., NVD, increased salivation).

A single dose of the formulation comprising 60 mg of oprozomib to a dog produces peak plasma concentration (Cₘₐₓ) of oprozomib of 66.4 ng/mL (having a standard deviation of 73.3).

The administration of the formulation to a dog produces an area under the concentration time curve to the last time point (AUC) of oprozomib of 28.6 ng*hr/mL, having standard deviation of 18.6).

### Example 7. Oprozomib Dosing Regimen

Dogs were dosed once a week on a one time basis, e.g., Day 1 and Day 8. Patients are expected to be administered oprozomib formulated in a tablet form according to either a QD×2 treatment schedule or QD×2 weekly treatment schedule. As used herein, “QD×2” means that patients receive oprozomib tablets once daily on days 1-2 of a 7-day treatment schedule. Patients may be administered oprozomib formulated in a tablet where the patient receives oprozomib on days one through 2 of a seven day treatment schedule.

The formulations of oprozomib may be administered with or without food; however, the formulations are preferably administered with a low fat meal.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Accordingly, other embodiments are within the scope of the following claims.

All of the above-cited references and publications are hereby incorporated by reference.

What is claimed is:

1. An immediate release formulation comprising an amount of oprozomib, or a pharmaceutically acceptable salt thereof; in which equal or more than about 80% of oprozomib, or a pharmaceutically acceptable salt thereof, is released after about 30 minutes as determined by UV under the following dissolution conditions:

| | |
|---|---|
| Dissolution medium | About 0.01N Hydrochloric acid (HCl) |
| Media volume | About 900 mL |
| Temperature | 37 ± 0.5° C. |
| Apparatus | USP No. 2 (Paddles) |
| Speed | 50 rpm through 60 minutes and 250 rpm for an |
| | additional 30 minutes |
| Sampling Time | About 15, 30, 45, 60 and 90 minutes |
| Infinity Point | About 30 min after last sample |
| Sampling Volume | About 10 mL. |

2. The formulation of claim 1, wherein the formulation provides a reduced incidence or severity of one or more gastrointestinal (GI) side effects.

3. The formulation of claim 2, wherein the side effects include emesis.

4. The formulation of claim 2, wherein the side effects include increased salivation.

5. The formulation of claim 1, wherein the formulation is in a form suitable for oral administration.

6. The formulation of claim 1, wherein the formulation provides oprozomib with time to peak plasma concentrations of from about 30 to about 120 minutes.

7. The formulation of claim 6, wherein the formulation provides oprozomib with time to peak plasma concentrations of from about 30 to about 60 minutes.

8. The formulation of claim 6, wherein the formulation provides oprozomib with time to peak plasma concentrations of about 60 minutes.

9. The formulation of claim 1, wherein the formulation comprises from about 5 weight percent to about 95 weight percent of oprozomib, or a pharmaceutically acceptable salt thereof.

10. The formulation of claim 9, wherein the formulation comprises from about 20 weight percent to about 50 weight percent of oprozomib, or a pharmaceutically acceptable salt thereof.

11. The formulation of claim 9, wherein the formulation comprises about 33 weight percent of oprozomib, or a pharmaceutically acceptable salt thereof.

12. The formulation of claim 9, wherein the formulation comprises from about 10 weight percent to about 40 weight percent of oprozomib, or a pharmaceutically acceptable salt thereof.

13. The formulation of claim 9, wherein the formulation comprises from about 20 weight percent to about 33 weight percent of oprozomib, or a pharmaceutically acceptable salt thereof.

14. The formulation of claim 1, wherein the formulation comprises about 50 milligrams of oprozomib, or a pharmaceutically acceptable salt thereof.

15. The formulation of claim 1, wherein the formulation comprises about 100 milligrams of oprozomib, or a pharmaceutically acceptable salt thereof.

16. The formulation of claim 1, wherein the formulation comprises about 200 milligrams of oprozomib, or a pharmaceutically acceptable salt thereof.

17. The formulation of claim 1, wherein the formulation comprises about 400 milligrams of oprozomib, or a pharmaceutically acceptable salt thereof.

18. The formulation of claim 1, wherein the formulation comprises from about 20 weight percent to about 40 weight percent of oprozomib, or a pharmaceutically acceptable salt thereof; and about 25 milligrams of oprozomib, or a pharmaceutically acceptable salt thereof.

19. The formulation of claim 1, wherein the formulation comprises from about 20 weight percent to about 40 weight percent of oprozomib, or a pharmaceutically acceptable salt thereof; and about 50 milligrams of oprozomib, or a pharmaceutically acceptable salt thereof.

20. The formulation of claim 1, wherein the formulation comprises from about 20 weight percent to about 40 weight percent of oprozomib, or a pharmaceutically acceptable salt thereof; and about 100 milligrams of oprozomib, or a pharmaceutically acceptable salt thereof.

21. The formulation of claim 1, wherein the formulation comprises from about 25 weight percent to about 60 weight percent of oprozomib, or a pharmaceutically acceptable salt thereof; and about 400 milligrams of oprozomib, or a pharmaceutically acceptable salt thereof.

22. The formulation of claim 1, wherein the formulation comprises about 33.33 weight percent of oprozomib, or a pharmaceutically acceptable salt thereof; and about 25 milligrams of oprozomib, or a pharmaceutically acceptable salt thereof.

23. The formulation of claim 1, wherein the formulation comprises about 33.33 weight percent of oprozomib, or a pharmaceutically acceptable salt thereof; and about 50 milligrams of oprozomib, or a pharmaceutically acceptable salt thereof.

24. The formulation of claim 1, wherein the formulation comprises about 33.33 weight percent of oprozomib, or a pharmaceutically acceptable salt thereof; and about 200 milligrams of oprozomib, or a pharmaceutically acceptable salt thereof.

25. The formulation of claim 1, wherein the oprozomib, or pharmaceutically acceptable salt thereof, is a crystalline solid.

26. The formulation of claim 1, wherein the oprozomib, or pharmaceutically acceptable salt thereof, is an amorphous solid.

27. The formulation of claim 1, wherein the formulation further comprises one or more fillers.

28. The formulation of claim 27, wherein the one or more fillers is selected from microcrystalline cellulose and lactose monohydrate.

29. The formulation of claim 1, wherein the formulation further comprises one or more disintegrants.

30. The formulation of claim 29, wherein the one or more disintegrants is selected from croscarmellose sodium.

31. The formulation of claim 1, wherein the formulation further comprises one or more lubricants.

32. The formulation of claim 31, wherein the one or more lubricants is magnesium stearate.

33. The formulation of claim 1, wherein the formulation further comprises one or more desiccants.

34. The formulation of claim 33, wherein the one or more desiccants is silicon dioxide.

35. The formulation of claim 34, wherein the silicon dioxide is porous.

36. A formulation, wherein the formulation comprises:
i) from about 20 to about 35 weight percent oprozomib, or a pharmaceutically acceptable salt thereof;
ii) from about 30 to about 70 weight percent one or more fillers;
iii) from about 0.01 to about 2 weight percent one or more lubricants; and
iv) from about 3.5 to about 5 weight percent one or more disintegrants.

37. The formulation of claim 36, wherein the formulation comprises from about 0.10 to about 2 weight percent one or more desiccants.

38. The formulation of claim 37, wherein the one or more desiccants is silicon dioxide.

39. The formulation of claim 38, wherein the silicon dioxide is porous.

40. The formulation of claim 36, wherein the formulation is a solid dosage form.

41. The formulation of claim 40, wherein the solid dosage form is a tablet.

42. The formulation of claim 36, wherein the formulation comprises one or more coatings.

43. The formulation of claim 36, wherein the tablet has a thickness of from about 2.5 millimeters to about 7 millimeters.

44. The formulation of claim 36, wherein the tablet has a hardness of from about 1.00 kp to about 25.00 kp.

45. The formulation of claim 36, wherein equal to or greater than about 80% of oprozomib, or a pharmaceutically acceptable salt thereof, is released within about 30 minutes.

46. The formulation of claim 36, wherein equal to or greater than about 80% of the oprozomib, or a pharmaceutically acceptable salt thereof, is released within about 45 minutes.

47. The formulation of claim 36, wherein equal to or greater than about 80% of the oprozomib, or a pharmaceutically acceptable salt thereof, is released within about 60 minutes.

48. The formulation of claim 36, wherein a single dose of the formulation comprising about 60 mg of oprozomib to a dog produces peak plasma concentration (Cₘₐₓ) of oprozomib of 66.4 ng/mL (having a standard deviation of 73.3).

49. The formulation of claim 36, wherein administration of the formulation to a dog produces an area under the concentration time curve to the last time point (AUC) of oprozomib of 28.6 ng*hr/mL. (having a standard deviation of 18.6).

50. The formulation of claim 36, wherein the formulation is stable upon actual or simulated storage under open conditions at 25° C./60% relative humidity for at least 1 month.

51. The formulation of claim 36, wherein the formulation is stable upon actual or simulated storage under open conditions at 40° C./75% relative humidity for at least 1 month.

52. The formulation of claim 1, wherein the formulation provides a reduced incidence or severity of one or more of nausea, vomiting and diarrhea.

53. The formulation of claim 36, wherein the formulation provides a reduced incidence or severity of one or more of nausea, vomiting and diarrhea.

54. The formulation of claim 36, wherein the formulation is prepared by dry granulation.

55. A method for treating a disease or condition selected from the group consisting of cancer, autoimmune disease, graft or transplant-related condition, neurodegenerative disease, fibrotic-associated condition, ischemic-related conditions, infection (viral, parasitic or prokaryotic) and diseases associated with bone loss, the method comprising administering a formulation as claimed in claim 1.

56. The method of claim 55, wherein the disease or condition is cancer.

55. The method of claim 54, wherein the cancer is selected from multiple myeloma, Waldenström's macroglobulinemia, chronic lymphocytic leukemia, and myelodysplastic syndromes.

57. A method for treating a disease or condition selected from the group consisting of cancer, autoimmune disease, graft or transplant-related condition, neurodegenerative disease, fibrotic-associated condition, ischemic-related conditions, infection (viral, parasitic or prokaryotic) and diseases associated with bone loss, the method comprising administering a formulation as claimed in claim 36.

58. The method of claim 57, wherein the disease or condition is cancer.

59. The method of claim 58, wherein the cancer is selected from multiple myeloma, Waldenström's macroglobulinemia, chronic lymphocytic leukemia, and myelodysplastic syndromes.
